SEQUENCE LISTING (25 sequences)

SEQ ID NO: 1 - DNA, 1277 bp, Mus musculus
aaggcgggcgaggcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgacagccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccgatcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgcgaggaaagcggtcccccagccgcgaagcgaggagggcgatgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctcgagaccaggcctccccgcaaagcacggagcaggacaaggagcgcctgcgtttccagttcttagagcagaagtacggctactatcactgcaaggactgcaaaatccggtgggagagcgcctatgtgtggtgtgtgcagggcaccagtaaggtgttacttcaaacagttctgccgagtgtgtgagaaatcctacaacccttacagagtggaggacatcacctgtcaaagttgtaaaagaactagatgtgcctgcccagtcagatttcgccacgtggaccctaaacgcccccatcggcaagacttgtgtgggagatgcaaggacaaacgcctgtcctgcgacagcaccttcagcttcaaatacatcatttagtgagagtcgaaaacgtttctgctagatggggctaatggaatggacaagtgagctttctcccctcttcacctcttccctttccaaattcttcatgacagacagtgttacttggatataaagcctgtgaataaaaggtattgcaaacaaaaaaaaaaaaaaaaaa

SEQ ID NO: 2 - protein, 361 aa, Mus musculus
MFPASTFHPCPHPYPQATKAGDGWRFGARGCRPAPPSFLPGYRQLMAAEYVDSHQRAQLMALLSRMGPRSVSSRDAAVQVNPRRDASVQCSLGRRTLQPAGCRASPDARSGSCQPRGHAGAGRSPRSWQTVAPFSSVTFCGLSSSLEVAGGRQTPTKGEGSPASSGTREPEPREVAARKAVPQPRSEEGDVQAAGQAGWEQQPPPEDRNSVAAMQSEPGSEEPCPAAEMAQDPGDSDAPRDQASPQSTEQDKERLRFQFLEQKYGYYHCKDCKIRWESAYVWCVQGTSKVYFKQFCRVCEKSYNPYRVEDITCQSCKRTRCACPVRFRHVDPKRPHRQDLCGRCKDKRLSCDSTFSFKYII

SEQ ID NO: 3 - DNA, 1817 bp, Mus musculus
gtcacagctttcccctgcccgaatatggtgatctgtctccattgtccagatcaggatgattctttagaagaagtcacagaggaatgctattccccacccaccctccagaacctggcaattcagagtctactgagggatgaggccttggccatttctgctctcacggacctgccccagagtctgttcccagtaatttttgaggaggccttcactgatggatatatagggatcttgaaggccatgatacctgtgtggcccttcccatacctttctttaggaaagcagataaataattgcaacctggagactttgaaggctatgcttgagggactagatatactgcttgcacaaaaggttcaaaccagtaggtgcaaactcagagtaattaattggagagaagatgacttgaagatatgggctggatcccatgaaggtgaaggcttaccagatttcaggacagagaagcagccaattgagaacagtgctggctgtgaggtgaagaaagaattgaaggtgacgactgaagtccttcgcatgaagggcagacttgatgaatctaccacatacttgttgcagtgggcccagcagagaaaagattctattcatctattctgtagaaagctactaattgaaggcttaaccaaagcctcagtgatagaaatcttcaaaactgtacacgcagactgtatacaggagcttatcctaagatgtatctgcatagaagagttggcttttcttaatccctacctgaaactgatgaaaagtcttttcacactcacactagatcacatcataggtaccttcagtttgggtgattctgaaaagcttgatgaggagacaatattcagcttgatttctcaacttcccacactccactgtctccagaaactctatgtaaatgatgtcccttttataaaaggcaacctgaaagaatacctcaggtgcctgaaaaagcccttggagacactttgcatcagtaactgtgacctctcacagtcagacttggattgcctgccctattgcctgaatatttgtgaactcaaacatctgcatattagtgatatatatttatgtgatttactccttgagcctcttggttttctccttgagagagttggagataccctgaaaaccctggaattggattcatgttgtatagtggactttcagttcagtgccttgctgcctgccctaagccaatgttctcacctcagagaggtcactttctatgataatgatgtttctctgcctttcttgaaaacaacttctacaccacacagccctgctgagtcagctgatctatgagtgttaccctgcccctctagagtgctatgatgacagtggtgtaatactaacacacagattagaaagtttttgtcctgagcttctggatatactgagagccaaaagacagctccatagtgtctcctttcaaacaaccaaatgctctaaatgtggtgggtgctacatttatgatcggcatacccaatgttgccgttttgtggaactactataagcttgattgtgaaactgagaaatagaaacttagtattggggactgatgaaatcctaagtgaatgtccactgctaaatggagcatgaaaatgtcaatcacctaaaagtctgagatacacaggaaagtcaataacttcctctgagctggtgaatggatgttgcatctgtagaaagtatcaagcacttgtagtttgaatgtgttacaatagaagcaccattttatgagactggcccaatctgttgactgcatacaataaatctgttgacttattaaatttttaaaaaaaaaaaaaaaaaaaaaa

SEQ ID NO: 4 - protein, 426 aa, Mus musculus
MVICLHCPDQDDSLEEVTEECYSPPTLQNLAIQSLLRDEALAISALTDLPQSLFPVIFEEAFTDGYIGILKAMIPVWPFPYLSLGKQINNCNLETLKAMLEGLDILLAQKVQTSRCKLRVINWREDDLKIWAGSHEGEGLPDFRTEKQPIENSAGCEVKKELKVTTEVLRMKGRLDESTTYLLQWAQQRKDSIHLFCRKLLIEGLTKASVIEIFKTVHADCIQELILRCICIEELAFLNPYLKLMKSLFTLTLDHIIGTFSLGDSEKLDEETIFSLISQLPTLHCLQKLYVNDVPFIKGNLKEYLRCLKKPLETLCISNCDLSQSDLDCLPYCLNICELKHLHISDIYLCDLLLEPLGFLLERVGDTLKTLELDSCCIVDFQFSALLPALSQCSHLREVTFYDNDVSLPFLKTTSTPHSPAESADL

SEQ ID NO: 5 - DNA, 1018 bp, Mus musculus
gccatattgaggacctgcagtagaggtggaacccatgactggcagcgcaaacacagtgataacagctgagctccaagcaaggacccaggaccttgcctcaccacagacataatctttccccacaacacctccaccaagccgccctgtaaatcgacatgagtcgccacagcaccagcagcgtgaccgaaaccacagcaaaaaacatgctctggggtagtgaactcaatcaggaaaagcagacttgcacctttagaggccaaggcgagaagaaggacagctgtaaactcttgctcagcacgatctgcctgggggagaaagccaaagaggaggtgaaccgtgtggaagtcctctcccaggaaggcagaaaaccaccaatcactattgctacgctgaaggcatcagtcctgcccatggtcactgtgtcaggtatagagctttctcctccagtaacttttcggctcaggactggctcaggacctgtgttcctcagtggcctggaatgttatgagacttcggacctgacctgggaagatgacgaggaagaggaggaagaggaggaggaagaggatgaagatgaggatgcagatatatcgctagaggagatacctgtcaaacaagtcaaaagggtggctccccagaagcagatgagcatagcaaagaaaaagaaggtggaaaaagaagaggatgaaacagtagtgaggcccagccctcaggacaagagtccctggaagaaggagaaatctacacccagagcaaagaagccagtgaccaagaaatgacctcatcttagcatcttctgcgtccaaggcaggatgtccagcagctgtgttttggtgcaggtgtccagccccaccaccctagtctgaatgtaataaggtggtgtggctgtaaccctgtaacccagccctccagtttccggaggtttttggtgaagagcccccagcaagttcgcctagggccacaataaaatttgcatgatcaggaaaaaaaaaaaaaaaaaaaaaaaaaaaaaaaaaaa

SEQ ID NO: 6 - protein, 207 aa, Mus musculus
MSRHSTSSVTETTAKNMLWGSELNQEKQTCTFRGQGEKKDSCKLLLSTICLGEKAKEEVNRVEVLSQEGRKPPITIATLKASVLPMVTVSGIELSPPVTFRLRTGSGPVFLSGLECYETSDLTWEDDEEEEEEEEEEDEDEDADISLEEIPVKQVKRVAPQKQMSIAKKKKVEKEEDETVVRPSPQDKSPWKKEKSTPRAKKPVTKK

SEQ ID NO: 7 - DNA, 214 bp, Mus musculus
acagcagaggtgatgctcagaaatcaagttttaacagagggccaggtgcttctagagtaggaggggattgcacacctccccaccccctcctctttcccaggcttcttaacagcctgctgtgggaagctgacccttagatggagccctgaagccatattgaggacctgcagtagaggtggaacccatgactggcagcgcagtaagcttgagcagg

SEQ ID NO: 8 - DNA, 194 bp, Mus musculus
ctttgcattactcagaacacagtgataacagctgagctccaagcaaggacccaggaccttgcctcaccacagacataatctttccccacaacacctccaccaagccgccctgtaaatcgacatgagtcgccacagcaccagcagcgtgaccgaaaccacagcaaaaaacatgctctggggtaagggctaaggct

SEQ ID NO: 9 - DNA, 116 bp, Mus musculus
gtcttcgctgtgcaggtagtgaactcaatcaggaaaagcagacttgcacctttagaggccaatgcgagaagaaggacagctgtaaactcttgctcagcacggtgggtgtctcccaa

SEQ ID NO: 10 - DNA, 144 bp, Mus musculus
catcacctttctcagatctgcctgggggagaaagccaaagaggaggtgaaccgtgtggaagtcctctcccaggaaggcagaaaaccaccaatcactattgctacgctgaaggcatcagtcctgcccatggtgagtcttctctcc

SEQ ID NO: 11 - DNA, 124 bp, Mus musculus
agaagggggacacaggtcactgtgtcaggtatagagctttctcctccagtaacttttcggctcaggactggctcaggacctgtgttcctcagtggcctggaatgttatggtaagttgtagccta

SEQ ID NO: 12 - DNA, 182 bp, Mus musculus
ggctacccattccagagacttcggacctgacctgggaagatgacgaggaagaggaggaagaggaggaggaagaggatgaagatgaggatgcagatatatcgctagaggagatacctgtcaaacaagtcaaaagggtggctccccagaagcagatgagcatagcaaaggtggggggaaaagaa

SEQ ID NO: 13 - DNA, 71 bp, Mus musculus
tggtttttgttccagaaaaagaaggtggaaaaagaagaggatgaaacagtagtgaggtaattcatgcagtt

SEQ ID NO: 14 - DNA, 64 bp, Mus musculus
ctattccctttccaggcccagccctcaggacaagagtccctggaagaaggtgagcaataagaag

SEQ ID NO: 15 - DNA, 364 bp, Mus musculus
ctcttatctgcacaggagaaatctacacccagagcaaagaagccagtgaccaagaaatgacctcatcttagcatcttctgcgtccaaggcaggatgtccagcagctgtgttctggtgcaggtgtccagccccaccaccctagtctgaatgtaataaggtggtgtggctgtaaccctgtaacccagccctccagtttccggaggtttttggtgaagagcccccagcaagttcgcctagggccacaataaaatttgcatgatcaggacctccctctgcctccccctccctggatgggtctcctcgctgctgcgatagctcatgtgcccagcagagggcaaccacgagcaagaaaccagccccatgt

SEQ ID NO: 16 - DNA, 924 bp, Human
cagcccgcttctctgcccggagccatgaatctcagtagcgccagtagcacggaggaaaaggcagtgacgaccgtgctctggggctgcgagctcagtcaggagaggcggacttggaccttcagaccccagctggaggggaagcagagctgcaggctgttgcttcatacgatttgcttgggggagaaagccaaagaggagatgcatcgcgtggagatcctgcccccagcaaaccaggaggacaagaagatgcagccggtcaccattgcctcactccaggcctcagtcctccccatggtctccatggtaggagtgcagctttctcccccagttactttccagctccgggctggctcaggacccgtgttcctcagtggccaggaacgttatgaagcatcagacctaacctgggaggaggaggaggaagaagaaggggaggaggaggaagaggaagaggaagatgatgaggatgaggatgcagatatatctctggaggagcaaagccctgtcaaacaagtcaaaaggctggtgccccagaagcaggcgagcgtggctaagaaaaaaaagctggaaaaagaagaagaggaaataagagccagcgttagagacaagagccctgtgaaaaaggccaaagccacagccagagccaagaagccaggattcaagaaatgaggagccacgccttggggggcacggtgcaaagtgggccttccctgggctgtgctgcaggcacagggtgcccctgtccagcccctccacctgtgtctgaatgcaacaggggtgttgcgggggcaacatgagagcccctcacccccaactctccactttcaggaggcccccagtgaagagccccacctcggggtcacaataaagttgcctggtcaggaaaaaaaaaaaaaaaaaaacgtttgcggccgcaagcttatg

SEQ ID NO: 17 - protein, 214 aa, Human
MNLSSASSTEEKAVTTVLWGCELSQERRTWTFRPQLEGKQSCRLLLHTICLGEKAKEEMHRVEILPPANQEDKKMQPVTIASLQASVLPMVSMVGVQLSPPVTFQLRAGSGPVFLSGQERYEASDLTWEEEEEEEGEEEEEEEEDDEDEDADISLEEQSPVKQVKRLVPQKQASVAKKKKLEKEEEEIRASVRDKSPVKKAKATARAKKPGFKK

SEQ ID NO: 18 - DNA, 814 bp, mus musculus
ggcgggcgaggcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgacagccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccgatcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgcgaggaaagcggtcccccagccgcgaagcgaggagggcgatgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctcgagaccaggcctccccgcaaagcacggagcaggacaaggagcgcctgcgtttccaggtgaggccagcctga

SEQ ID NO: 19 - DNA, 123 bp, mus musculus
taccctgctgttcagttcttagagcagaagtacggctactatcactgcaaggactgcaaaatccggtgggagagcgcctatgtgtggtgtgtgcagggcaccagtaaggtaagagacaccgtg

SEQ ID NO: 20 - DNA, 105 bp, mus musculus
tctttctcctcgcaggtgtacttcaaacagttctgccgagtgtgtgagaaatcctacaacccttacagagtggaggacatcacctgtcaagtaaaccaaacgttt

SEQ ID NO: 21 - DNA, 305 bp, mus musculus
actccgatttttcagagttgtaaaagaactagatgtgcctgcccagtcagacttcgccacgtggaccctaaacgcccccatcggcaagacttgtgtgggagatgcaaggacaaatgcttgtcctgcgacagcaccttcagcttcaaatacatcatttagtgagagtacgaaacgtttctgctagatggggctaatggaatggacaagtgagctttctcccctcttccctcttcccatttccaaattcttcatgacagacagtgttacttggatataaagcctgtgaataaaaggtattgcaaaca

SEQ ID NO: 22 - DNA, 809 bp, mus musculus
ggcgggcgaggcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgacagccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccggtcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgtgaggaaagcggtcccccagccgcgaagcgaggagggcgacgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctccccgcaaagcaccataccctgcagcaggacaaggagctcctgcgtttccaggtgaggccagcctgg

SEQ ID NO: 23 - DNA, 123 bp, mus musculus; identical to SEQ ID NO: 19

SEQ ID NO: 24 - DNA, 105 bp, mus musculus
tctttctcctcgtaggtgtacttcaaacagttctgccgagtgtgtgagaaatcctacaacccttacagagtggaggacgtcacctgtcaagtaaaccaaacgttt

SEQ ID NO: 25 - DNA, 375 bp, mus musculus
gctctgagttttcagagttgtaaaggaactagatgtgcctgcccagtcagacttcgccacgtggaccctaaacgcccccatcggcaagacttgtgtgggaagatgtgcctgcccagtcagacctcgccacgtgtaccttagacgcccccatcagcaagacttgtgtgagagatgcaaggacaaacgcctgtcctgcgacagcaccgtcagcttcaaatacatgatttagtgagagtcgaaaacgtttctgctagatggggctaatggaatggacaagtgagctttctcccctcttcacctcttccctttccaaattcttcatgacagacagtgttacttggatataaagcctgtgaataaaaggtattgcaaaca

(19) United States
(12) Patent Application Publication
(10) Pub. No.: US 2002/0042926 A1
(43) Pub. Date: Apr. 11, 2002

(54) Ovary-specific genes and proteins

(76) Inventors: Martin M. Matzuk, Pearland, TX (US); Pei Wang, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 09/844,864

(22) Filed: Apr. 27, 2001

Related U.S. Application Data
(63) Continuation-in-part of application No. PCT/US99/25209, filed on Oct. 28, 1999.
(60) Provisional application No. 60/106,020, filed on Oct. 28, 1998.

(51) Int. Cl. A01K 67/027; C07H 21/04; C12N 9/00

(52) U.S. Cl. 800/18; 536/23.2; 435/183

(57) ABSTRACT

Ovary-specific proteins O1-180, O1-184 and O1-236, polynucleotides encoding them, antibodies which are immunoreactive with them and vectors and host cells containing O1-180, O1-184 or O1-236 and transgenic mice comprising disruptions of those genes are provided. Also provided are methods for detecting cell proliferative or degenerative disorders of ovarian origin and which are associated with O1-180, O1-184 or O1-236 and for creating transgenic mice comprising disruptions of those genes. Further provided are methods for the evaluation of potential contraceptives using the proteins of the invention, as well as methods for the screening for genetic mutations in signaling pathways that are associated with some forms of human infertility or gynecological cancers, also using the proteins/mRNAs/genes of the invention. The proteins/mRNAs/genes of the invention may also be used as markers for identifying primary and metastatic neoplasms of ovarian origin and as indicators of developmental anomalies in prenatal screening procedures. Furthermore, assays of the proteins/mRNAs/genes of the invention can be used in diagnostic assays for detecting forms of infertility and other diseases, including germ cell tumors and polycystic ovary syndrome. The proteins of the invention may be useful targets for in vitro fertilization procedures or in enhancing the number of eggs that can be retrieved from the human donor, e.g., in enhancing the success rate.

This application is a continuation-in-part application of International Application Number PCT/US99/25209 filed Oct. 28, 1999, which is an international application claiming priority to U.S. Provisional Application No. 60/106,020 filed Oct. 28, 1998.

### BACKGROUND OF THE INVENTION

1. Field of the Invention

The present invention relates generally to ovary-specific genes and the proteins they encode.

2. Description of Related Art

Reproductive development and function are complex processes involving both genetically-determined and physiological events. Identification of the critical protein products of genes involved in these processes is necessary to characterize how these processes are regulated. Although important molecular events occur during the early phases of mammalian oogenesis and folliculogenesis, to date, few “candidate” regulatory molecules have been identified and characterized thoroughly. Several studies have suggested that both endocrine factors, such luteinizing hormone (LH) and follicle stimulating hormone (FSH) from the pituitary, as well as paracrine factors secreted from the oocyte influence folliculogenesis. FSH and LH are known to bind to granulosa and thecal cells which in turn are required for oocyte growth and maturation and maintenance of oocyte meiotic competence. Likewise, oocytes may secrete factors which are necessary for normal granulosa cell and thecal cell function. Because oocyte growth is coordinated with the development and growth of the surrounding somatic cells (i.e., granulosa cells initially and thecal cells later), understanding the molecular events at early stages will give important clues about the paracrine factors mediating the reciprocal interactions between oocytes and somatic cells, the development of competence for trophic hormone stimulation, and the process of follicular recruitment.

Disruption of the hypothalamic-pituitary-gonadal reproductive axis by administration of steroids containing synthetic estrogens and progestins has been one of the oldest methods of hormonal contraception However, the latest report of the Institute of Medicine emphasizes the importance of developing strategies for new contraceptives. According to the report, some of the long-term contraceptive strategies for women include inhibition of ovulation, prevention of fertilization, or blocking of implantationof a fertilized egg into the uterine lining. Furthermore, infertility affects ˜15% of couples, and in ˜40% of the cases, the female is believed to be the sole cause of the infertility. Thus, it is critical to identify novel ovary-specific gene products which could be potential targets for new contraceptive agents.

One function of the ovary is to produce an oocyte that is fully capable of supplying all the necessary proteins and factors for fertilization and early embryonic development. Oocyte-derived mRNA and proteins are necessary for the removal of the sperm nuclear envelope, the decondensation of the sperm nucleus (including the removal of protamines), the assembly of histones on the sperm DNA and chromatin condensation, the completion of oocyte meiotic maturation and extrusion of the second polar body, the formation of male and female pronuclei, the fusion of male and female pronuclei, the replication of DNA, and the initiation of zygote and early embryonic cleavages [reviewed in (Perreault, 1992)]. Oocyte-derived factors are necessary since the sperm contains mainly DNA (i.e., no cytoplasm or nucleoplasm), and many of the factors necessary for early post-fertilization events in mammals are acquired during oocyte meiotic maturation (McLay and Clarke, 1997). These oocyte proteins are predicted to be highly conserved through evolution since oocytes can efficiently remodel heterologous sperm or somatic cell nuclei into pronuclei (Perreault, 1992). Although histones are involved in the modification of the sperm chromatin to resemble that of a somatic cell, the other non-histone proteins involved in these processes are unknown in mammals. In Xenopus laevis, a key factor in sperm decondensation is nucleoplasmin which was isolated and cloned over a decade ago (Burglin et al., 1987; Dingwall et al., 1987). Sperm chromatin decondensation occurs after a spermatotozoon enters an egg. In Xenopus laevis, although reduction of the protamine disulfide bonds by ooplasmic glutathione is important, nucleoplasmin (also called nucleoplasmin A or Xnpm2) is necessary and sufficient to initiate the decondensation of sperm nuclei (Philpott et al., 1991). Nucleoplasmin, an acidic, thermostable protein, is the most abundant protein in the nucleus of Xenopus laevis oocytes and eggs, making up 7-10% of the total nuclear protein (Krohne and Franke, 1980a; Mills et al., 1980). After germinal vesicle breakdown, nucleoplasmin [present in the egg nucleoplasm but not bound to DNA (Mills et al., 1980)], is released into the ooplasm where it functions to bind protamines tightly and strip them from the sperm nucleus within 5 minutes of sperm entry, resulting in sperm decondensation (Ohsumi and Katagiri, 1991; Philpott and Leno, 1992; Philpott et al., 1991). This process allows egg histones to subsequently bind the sperm DNA. Immunodepletion of nucleoplasmin from egg extracts prevents sperm decondensation (Philpott et al., 1991). Direct interaction of nucleoplasmin with protamine was observed in in vitro experiments, which suggest that the nucleoplasmin is bound to protamine in a 1:1 ratio and that the polyglutamic acid tract in nucleoplasmin plays a critical role for binding to protamine (Iwata et al., 1999). Interestingly, injection of sperm DNA into oocyte nuclei, male or female pronuclei of fertilized eggs, or nuclei of 2 cell embryos leads to sperm decondensation (Maeda et al., 1998), suggesting that nucleoplasmin is functional at all of these stages. Nucleoplasmin can also interact with histones as a pentamer (Earnshaw et al., 1980; Laskey et al., 1993). Nucleoplasmin binds specifically to histones H2A and H2B and along with the proteins N1/N2 that bind histones H3 and H4, can promote nucleosome assembly onto DNA (Dilworth et al., 1987; Laskey et al., 1993). Thus, these observations suggest that at fertilization, the oocyte-derived nucleoplasmin interacts with the sperm nucleus, dissociates the sperm protamines from the DNA, interacts with histones, and brings about sperm minichromatin assembly (Laskey et al., 1993; Philpott et al., 1991). Although “ubiquitous” proteins with low homology to nucleoplasmin have been cloned in mammals and Drosophila (Chan et al., 1989; Crevel et al., 1997; Ito et al., 1996; MacArthur and Shackleford, 1997b; Schmidt-Zachmann and Franke, 1988), an oocyte-equivalent ortholog in mammals had not yet been identified.

In an effort to identify other novel ovarian-expressed genes that may play key functions in ovarian physiology, fertilization and early cleavage events, the inventors have used a subtractive hybridization approach. Several novel oocyte-expressed genes have been identified by the inventors which are important in regulating oogenesis, folliculogenesis, fertilization, and/or early embryogenesis. One of these oocyte-specific gene products, nucleoplasmin 2 (O1-236), is the mammalian ortholog of Xenopus laevis nucleoplasmin (Xnpm2)(Burglin et al., 1987; Dingwall et al., 1987). The 207 amino acid open reading frame of Npm2 demonstrated high homology to the family of proteins called nucleoplasmins or nucleophosmins (nomenclature designation=NPM in human, Npm in mouse, and Xnpm in Xenopus). Human nucleophosmin (NPM1 also called N038; accession # M23613) maps to human chromosome 5q35, encodes a 294 amino acid protein, and has orthologs in mouse (Npm 1, also called B23, accession # Q61937) and Xenopus laevis (Xnpm1 or N038 accession # X05496). Mouse nucleoplasmin/nucleophosmin homolog Npm3, which has been mapped to mouse chromosome 19, is 175 amino acids [accession # U64450, (MacArthur and Shackleford, 1997a)], and there is an apparent human NPM3 homolog (accession # AF081280). In contrast to Npm2, Npm1 and Npm3 are ubiquitously expressed genes, and the structure of the mouse Npm2 gene is considerably divergent compared to the mouse Npm3 gene (MacArthur and Shackleford, 1997a).

The Npm2 cDNA sequences have been used by the inventors to obtain the mouse Npm2 gene and the human NPM2 cDNA and gene and also map these genes. Mice lacking Npm2 have defects in fertility due to abnormalities in early post-fertilization cleavage events. The discovery of the mammalian homolog of the most abundant nuclear protein in Xenopus laevis oocytes and eggs (Krohne and Franke, 1980a; Mills et al., 1980) is important for a clear understanding of oogenesis, fertilization, and post-fertilization development in mammals and possibly also to define further oocyte factors which are necessary in mammalian cloning experiments.

Likewise, several studies have shown that phosphorylation of nucleoplasmin influences its function. Comparison of the forms of nucleoplasmin from the oocyte (i.e., in the ovary) versus egg (i.e., after ovulation and ready for fertilization) demonstrate dramatic differences in the level of phosphorylation. Xenopus laevis egg nucleoplasmin is substantially larger than the oocyte form, migrating ˜15,000 daltons larger on SDS-PAGE due to phosphorylation differences (Sealy et al., 1986). Nucleoplasmin has ˜20 phosphate groups/protein in the egg compared to <10 phosphate groups/proteins in the oocyte, and an egg kinase preparation can modify the oocyte nucleoplasmin so it resembles the egg form (Cotten et al., 1986). Functionally, this hyperphosphorylation of nucleoplasmin stimulates its nuclear transport (Vancurova et al., 1995) and also results in a more active form, leading to increased nucleosome assembly (Sealy et al., 1986) and sperm decondensation (Leno et al., 1996). A hyperphosphorylated form of nucleoplasmin is also present during the early stages of Xenopus laevis embryogenesis where it is believed to play some function during the rapid cell cycles and DNA replication (Burglin et al., 1987). The high percentage of serine and threonine residues in Npm2 and NPM2 suggest a similar role of phosphorylation of mammalian nucleoplasmin 2 in mammalian eggs. The functional importance of posttranslational modifications of Npm2 was highlighted by the inventors results that neither bacterial-produced recombinant (unphosphorylated) human and mouse Npm2 were able to decondense human or mouse sperm DNA (unpublished data). Phosphorylation could act to regulate when Npm2 acts, making it inactive until the critical time (i.e., fertilization). Although there are multiple putative kinase sites in both nucleoplasmin and Npm2, casein kinase II specifically interacts with nucleoplasmin and phosphorylates it, and an inhibitor of casein kinase II can block nuclear transport of Xenopus laevis nucleoplasmin (Vancurova et al., 1995). Interestingly, two of the predicted casein kinase II phosphorylation sites are conserved between nucleoplasmin (Ser¹²⁵ and Ser¹⁷⁷), Npm2 (Thr¹²³ and Ser¹⁸⁴), and NPM2 (Thr¹²⁷ and Ser¹⁹¹. Although other phosphorylation sites are likely important, a casein kinase II-Npm2 interaction in vivo could be predicted in mammals.

The basic functional unit within the ovary is the follicle, which consists of the oocyte and its surrounding somatic cells. Fertility in female mammals depends on the ability of the ovaries to produce Graafian follicles, which ovulate fertilizable oocytes at mid-cycle (Erickson and Shimasaki, 2000). This process, termed folliculogenesis, requires a precise coordinate regulation between extraovarian and intraovarian factors (Richards, et al., 1995). Compared to the knowledge of extraovarian regulatory hormones at the levels of the hypothalamus (i.e., GnRH) and anterior pituitary (i.e., FSH and LH), little is known about paracrine and autocrine factors within the ovaries, though oocyte-somatic cell communication has been long recognized as important (Falck, 1959). Accumulating evidence shows that factors secreted by the oocyte promote the proliferation of surrounding granulosa cells, and inhibit premature luteinization of these cells during folliculogenesis (El-Fouly et al., 1970; Channing, 1970). Oocyte factors have been implicated in controlling granulosa cell synthesis of hyaluronic acid, urokinase plasminogen activator (uPA), LH receptor, and steroids (El-Fouly et al., 1970; Nekola and Nalbandov, 1971; Salustri et al., 1985; Vanderhyden et al., 1993; Eppig et al., 1997a, b).

Several novel regulatory proteins have been recently discovered within oocytes. Growth differentiation factor 9 (GDF-9), a member of transforming growth factor β (TGF-β) superfamily, is one of the most important signaling factors. Oocyte expression of GDF-9 begins at the primary follicle stage, and persists through ovulation in the mouse (McGrath et al., 1995; Elvin et al., 2000). Female GDF-9-deficient mice are infertile due to a block of folliculogenesis at the type 3b (primary) follicle stage, accompanied by defects in granulosa cell growth and differentiation, theca cell formation, and oocyte meiotic competence (Dong et al., 1996; Carabatsos et al., 1998, Elvin et al, 1999A). The inventors have also reported that recombinant GDF-9 affects the expression of the genes encoding hyaluronic acid synthase 2 (Has2), cyclooxygenase 2 (Cox2), steroid acute regulatory protein (StAR), the prostaglandin E2 receptor EP2, LH receptor and uPA (Elvin et al., 1999B, Elvin et al., 2000).

To identify key proteins in the hypothalamic-pituitary-gonadal axis, we have previously generated several important knockout mouse models, including four which have ovarian defects. Mice deficient in gonadal/pituitary peptide inhibin have secondary infertility due to the onset of ovarian or testicular tumors which appear as early as 4 weeks of age (Matzuk, et al., 1992). Mice deficient in activin receptor type II (ActRII) survive to adulthood but display reproductive defects. Male mice show reduced testes size and demonstrate delayed fertility (Matzuk, et al. 1995). In contrast, female mice have a block in folliculogenesis at the early antral follicle stage leading to infertility. Consistent with the known role of activins in FSH homeostasis, both pituitary and serum FSH levels are dramatically reduced in these ActRII knockout mice. Female mice deficient in FSH, due to a mutation in the FSHβ gene, are infertile (Kumar et al., 1997). However, these mice have an earlier block in folliculogenesis prior to antral follicle formation. Thus, FSH is not required for formation of a multi-layer pre-antral follicle, but it is required for progression to antral follicle formation. Finally, growth differentiation factor 9 (GDF-9)-deficient mice have been used to determine at which stage in follicular development GDF-9 is required (Dong et al., 1996). Expression of GDF-9 mRNA is limited to the oocyte and is seen at the early one-layer primary follicle stage and persists through ovulation. Absence of GDF-9 results in ovaries that fail to demonstrate any normal follicles beyond the primary follicle stage. Although oocytes surrounded by a single layer of granulosa cells are present and appear normal histologically, no normal two-layered follicles are present. Follicles beyond the one-layer stage are abnormal, contain atypical granulosa cells, and display asymmetric growth of these cells. Furthermore, as determined by light and electron microscopy, a thecal cell layer does not form in these GDF-9-deficient ovaries. Thus, in contrast to kit ligand and other growth factors which are synthesized by the somatic cells and influence oocyte growth, GDF-9 functions in the reciprocal manner as an oocyte-derived growth factor which is required for somatic cell function. The novel ovary-specific gene products presented herein are expected to function in similar ways to regulate oogenesis and/or somatic cell function (e.g., folliculogenesis).

### SUMMARY OF THE INVENTION

The present invention provides three ovary-specific and oocyte-specific genes, O1-180, O1-184 and O1-236, the protein products they encode, fragments and derivatives thereof, and antibodies which are immunoreactive with these protein products. These genes and their protein products appear to relate to various cell proliferative or degenerative disorders, especially those involving ovarian tumors, such as germ cell tumors and granulosa cell tumors, or infertility, such as premature ovarian failure.

Thus, in one embodiment, the invention provides methods for detecting cell proliferative or degenerative disorders of ovarian origin and which are associated with O1-180, O1-184 or O1-236. In another embodiment, the invention provides method of treating cell proliferative or degenerative disorders associated with abnormal levels of expression of O1-180, O1-184 or O1-236, by suppressing or enhancing their respective activities.

The present invention provides key in vitro and in vivo reagents for studying ovarian development and function. The possible applications of these reagents are far-reaching, and are expected to range from use as tools in the study of development to therapeutic reagents against cancer. The major application of these novel ovarian gene products is to us them as reagents to evaluate potential contraceptives to block ovulation in women in a reversible manner. It will also be expected that these novel ovarian gene products will be useful to screen for genetic mutations in components of these signaling pathways that are associated with some forms of human infertility or gynecological cancers. In addition, depending on the phenotypes of humans with mutations in these genes or signaling pathways, we may consider using these novel ovarian gene products as reagent tools to generate a number of mutant mice for the further study of oogenesis and/or folliculogenesis. Such knockout mouse models will provide key insights into the roles of these gene products in human female reproduction and permit the use of these gene products as practical reagents for evaluation of new contraceptives.

### BRIEF DESCRIPTION OF THE DRAWINGS

FIG. 1 shows the 1276 base pair cDNA sequence of gene O1-180 (SEQ ID NO: 1).

FIG. 2 shows the 361 amino acid sequence that is coded for by gene O1-180 (SEQ ID NO: 2).

FIG. 3 shows the 1817 base pair cDNA sequence of gene O1-184 (SEQ ID NO: 3).

FIG. 4 shows the 426 amino acid sequence that is coded for by gene O1-184 (SEQ ID NO: 4).

FIG. 5 shows the 1019 base pair cDNA sequence of gene O1-236 (SEQ ID NO: 5).

FIG. 6 shows the 207 amino acid sequence that is coded for by gene O1-236 (SEQ ID NO: 6)

FIG. 7. Multi-tissue Northern blot analysis of ovary-specific genes. Northern blot analysis was performed on total RNA using O1-180, O1-184, and O1-236 probes. These gene products demonstrate an ovary-specific pattern (OV, ovary; WT, wild-type; −/−, GDF-9-deficient) as shown. The migration positions of 18S and 28S ribosomal RNA are indicated. All lanes had approximately equal loading as demonstrated using an 18S rRNA cDNA probe. Br, brain; Lu, lung; He, heart; St, stomach; Sp, spleen; Li, liver; SI, small intestine; Ki, kidney; Te, testes, Ut, uterus.

FIG. 8. In situ hybridization analysis of ovary-specific genes in mouse ovaries. In situ hybridization was performed using anti-sense probes to O1-180 (A, B), O1-184 (C,D) and O1-236 (E,F). A, C, and E are brightfield analysis of the ovaries. B, D, and F are darkfield analysis of the same ovary sections. All genes demonstrate specific expression in the oocyte beginning at the one layer primary follicle stage (small arrows) and continuing through the antral follicle stage (large arows). The “sense” probe does not detect a signal for any of these three ovary-specific genes (data not shown).

FIG. 9. In situ hybridization analysis of O1-236 in mouse ovaries. In situ hybridization was performed using probe O1-236 (partial Npm2 fragment). Brightfield analysis (A) and darkfield analysis (B) of the O1-236 mRNA in the same adult ovary sections. The probe demonstrates specific expression in all growing oocytes. Oocyte-specific expression is first seen in the early one layer primary follicle (type 3a), with higher expression in the one layer type 3b follicle and all subsequent stages including antral (an) follicles. The “sense” probe does not detect a signal for this oocyte-specific gene (data not shown).

FIG. 10. Npm2 cDNA representation. Schematic representation of the mouse Npm2 cDNA sequence (984 bp) and two of the clones isolated from the mouse ovary cDNA libraries. The original O1-236 probe (749 bp) is shown at the top and encompasses the entire Npm2 open reading frame. The open reading frame (solid box) is 621 bp and the 5′ UTR and 3′ UTR sequences (thin lines) are 155 bp and 205 bp, respectively. The polyA sequences are not depicted. Clone 236-1 was isolated from the wild-type ovary cDNA library and clone 236-3 was isolated from the GDF-9-deficient ovary cDNA library. Clone 236-3 (984 bp excluding polyA sequence) is 4 bp longer at the 5′ end and 1 bp longer at the 3′ end than clone 236-1 (979 bp excluding polyA sequences). Codon 36 of the open reading frame of both cDNAs is GGC (Glycine; FIG. 11) whereas the same codon of the 129SvEv gene is TGC (Cysteine; FIGS. 13A and 13B (SEQ ID NO: 7 through SEQ ID NO: 14)).

FIG. 11. Amino acid sequence conservation between mouse Npm2 and Xenopus laevis nucleoplasmin (Xnpm2). Using the NCB1 blast search tools, comparison of mouse Npm2 and Xnpm2 (accession # P05221) amino acid sequences reveals high identity (line connecting amino acids) and similarity (dots connecting amino acids). Spaces between the amino acids indicate gaps to aid in the alignment. Also identified are the conserved bipartite nuclear localization signal (bolded and underlined), the highly acidic “histone binding” region (boxed), and several conserved casein kinase II (CK2) and protein kinase C (PKC) phosphorylation sites (underlined and marked with “CK” or “PKC” with the serine or threonine in bold). Other predicted phosphorylation sites in either Npm2 or Xnpm2, which are not conserved, are not shown.

FIG. 12. Structure of the mouse Npm2 gene. Two overlapping recombinant λ clones (236-13 and 236-14), isolated from a mouse 129SvEv library, are shown at the top, and a schematic enlargement of the Npm2 gene is also depicted. Open boxes represent untranslated regions and solid black boxes represent protein coding regions. The 236-13 insert is ˜19.0 kb and 236-14 insert is ˜21.0 kb. The entire contig is ˜37 kb. All 9 exons of the Npm2 gene are encompassed on a single 6.9 kb Xbal (X) fragment as shown. The size of exons and introns are shown at the bottom. Abbreviations: B, BamH1; (B), predicted but unmapped BamH1; (N), NotI from phage cloning site.

FIGS. 13A and 13B. Mouse Npm2 gene (SEQ ID NO: 7 through SEQ ID NO: 14) and amino acid sequences. Uppercase letters represent sequence identity with the Npm2 cDNA sequences; non-transcribed 5′ and 3′ sequences and intron sequences are shown in lowercase. The predicted transcription initiation codon, the termination codon, and the polyadenylation signal sequence are all underlined. Numbers along the left side represent the amino acids. The underlined and bolded “T” in codon 36, the bolded “c” for amino acid 26, and the underlined and bolded “C: in the 3′ UTR sequence indicate differences between the cDNA and gene sequences. Arrows indicate where the O1-236 fragment initiates and ends in the cDNA sequence.

FIG. 14. Chromosomal localization of the mouse Npm2 gene. (Top) Map figure from the T31 radiation hybrid database at The Jackson Laboratory showing Chromosome 14 data. The map is depicted with the centromere toward the top. Distances between adjacent loci in centiRay3000 are shown to the left of the chromosome bar. The positions of some of the chromosome 14 MIT markers are shown on the right. Npm2 is positioned between D14Mit203 and D14Mit32. Missing typings were inferred from surrounding data where assignment was unambiguous. Raw data from The Jackson Laboratory were obtained from the Wold Wide Web address http://wwwjax.org/resources/documents/cmdata/rhmap/rh.html. (Bottom) Haplotype figure from the T31 radiation hybrid database at The Jackson Laboratory showing part of Chromosome 14 with loci linked to Npm2. Loci are listed in the best fit order with the most proximal at the top. The black boxes represent hybrid cell lines scoring positive for the mouse fragment and the white boxes represent cell lines scoring as negative. The grey box indicates an untyped or ambiguous line. The number of lines with each haplotype is given at the bottom of each column of boxes. Missing typings were inferred from surrounding data where assignment was unambiguous.

FIG. 15. Amino acid sequence conservation among Npm2, NPM2 and Xnpm2. Using the NCBI blast search tools and Megalign software, comparison of mouse (m) Npm2, human (h) Npm2, and Xenopus laevis Xnpm2 amino acid sequences reveals high identity (amino acids highlighted in blue). Spaces between the amino acids indicate gaps to aid in the alignment. Also identified are the conserved bipartite nuclear localization signal (red), the highly acidic histone and protamine binding region (red), and several conserved casein kinase II (CK2) and protein kinase C (PKC) phosphorylation sites (underlined and marked with “CK” or “PKC”). Other predicted phosphorylation sites in the nucleoplasmins, which are not conserved, are not shown.

FIG. 16. Analysis of Npm2 mRNA and Npm2 protein in mouse ovaries and early embryos. In situ hybridization was performed using probe O1-236 partial Npm2 cDNA fragment). Brightfield analysis (A) and darkfield analysis (B) of the O1-236 mRNA in the same adult ovary sections. The probe demonstrates specific expression in all growing oocytes. Oocyte-specific expression is first seen in the early one layer primary follicle (type 3a), with higher expression in the one layer type 3b follicle and all subsequent stages including antral (an) follicles. The “sense” probe does not detect a signal for this oocyte-specific gene (data not shown). (C) Immunohistochemistry of ovaries from a 5-week old mouse stained for Npm2 in the nuclei (bright red) of oocytes from type 3 (arrow) to antral follicles. (D) In preovulatory GVB oocytes induced by luteinizing hormone (hCG), Npm2 is evenly stained in the cytoplasm (arrow). An LH (hCG) unresponsive preantral follicle (upper right) continues to demonstrate an oocyte with Npm2 protein localized to the nucleus. (E) After fertilization, Npm2 begins to localize in the pronuclei; the formation of one pronucleus (arrow), is in the process of forming and some of Npm2 staining continues to be present in the cytoplasm of this early one cell embryo. (F) The pronuclei stain strongly in an advanced one cell embryo where very little Npm2 remains in the cytoplasm. Npm2 antibodies also specifically stain the nuclei of two cell (G) and eight cell (H) embryos.

FIG. 17. Gene targeting constructs for Npm2 and genotype analysis of offspring from heterozygote intercrosses. (A) The targeting strategy used to delete exon 2, exon 3, and the junction region of exon 4. PGK-hprt and MC1-tk expression cassettes are shown. Recombination were detected by Southern blot analysis using 5′ and 3′ probes. (B, BamH1; Bg, Bgl II; P, Pst I). (B) Southern blot analysis of genomic DNA isolated from mice generated from intercrosses of Npm2^{+/-} mice. The 3′ probe identifies the wild-type 7.5-kb band and the mutant 10.3-kb band when DNA was digested with Bgl II. (C) When DNA was digested with Pst 1, the exon 2 probe against only detected the wild-type 4.5-kb fragment.

FIG. 18. Histological analysis of ovaries from wild-type, Npm2^{+/-}, and Npm2^{-/+} mice. (A-D) Immunohistochemistry of ovaries from 6-week old mice stained for Npm2 in the nuclei (bright red) of oocytes (A and C for Npm2 ^{+/-} ovaries; B and D for Npm2^{-/-} ovaries). (E-F) PAS staining of ovaries from 12 week old mice wild-type (e) and Npm2^{-/-} (f) ovaries. Arrows show large antral follicles; “CL” denote corpora lutea.

FIG. 19. In vitro culture of eggs and fluorescent-labeling of DNA from fertilized eggs from Npm2^{-/-} and control mice. Eggs were isolated from the oviducts of immature mice after superovulation and cultured in vitro. Pictures were taken under a microscope at 24 and 48 hours of culture. (A, C) Most of the eggs from wild-type mice divided to form two cell embryos by 24 h; some of two cell embryos progressed to the four cell stage after 48 h of culture. (B, D) Very few eggs from Npm2^{-/-} mice cleaved into two cell embryos; no four cell embryos were detected after 48 hours of culture. Some developmentally abnormal or apparently apoptosed embryos from Npm2^{-/-} mice were detected. (E, F) Three Hoechst-stained fertilized eggs from wild-type mice contained 2 similar size pronuclei. The polar bodies (green arrowheads) around the edge of these fertilized eggs were also stained. (G-I) Non-decondensed sperm nuclei were strongly stained in the cytoplasm of the Npm2^{-/-} embryos (red arrows, G-I). The inserts in H and I show the condensed sperm heads at higher magnification. The maternal pronuclei are also labeled strongly in these Npm2-deficient embryos. (J) Two normal-appearing pronuclei were detected in some fertilized eggs derived from Npm^{-/-} females. A polar body (green arrowhead) is also obvious in this image.

FIG. 20. Localization of Oo1 in mouse ovaries. Expression of Oo1 in PMSG-treated wild-type (A and B) and GDF-9-deficient (C-F) ovaries was analyzed by in situ hybridization with a specific antisense probe. The expression of Oo1 gene was detectable at early primary follicle stage (type 3a) through ovulatory follicle stage, but not in primordial follicles in wild-type ovaries. In GDF-9-deficient ovaries, the follicle numbers was increased per unit volume due to the arrest of follicle development at primary follicle stage, more Oo1 positive signal were detected in each section. A, C and E, brightfield analysis of the ovaries; B, D and F, corresponding darkfield analysis of the same ovary sections. E and F are high power magnification of the same sections shown in C and D. The follicle classification method is based on Petersen and Peters (1968): type 2, primordial; type 3a, early primary; type 3b, late primary; type 8, preovulatory follicle.

FIG. 21. Structure of the Oo1 gene and Oo1 pseudogene. Diagrams representing the Oo1 pseudogene and the Oo1 gene are shown at the top along with unique restriction endonucleases sites which were important in constructing the linear map shown at the bottom Exons and introns are drawn to scale. Boxes denote exons, hatched regions denote protein coding portions and the solid regions denote the untranslated portions. Lines connecting boxes denote introns. Oo1ps: Oo1 pseudogene; Oo1: Oo1 gene; B: BamHI; S: SalI; X: XhoI;

FIGS. 22A and 22B. Comparison of Oo1 gene and Oo1 pseudogene. Sequences of exons, exon-intron boundaries and the size of each intron are shown. Different nucleotides between the two genes and consensus polyadenylation sequence are underlined. The translation start codon and stop codon are shown in bold. The consensus donor sequence in rodents is (C/A)AG/GTUAGT and the consensus acceptor sequence is YYYYYYYYYYNCAG/G (Y, pyrimidine; U, purine; N, any nucleotide) (Senapathy et al., 1990). Upper case: exon sequences; lower case: intron sequences.

FIG. 23. Maps of mouse chromosome 5, showing the position in centiMorgan (cM) of the marker best linked to Oo1 gene (A) and its related pseudogene (B) (data and maps generated at the Jackson Laboratory Bioinformatics Server).

FIG. 24. Gene targeting constructs for Oo1. The targeting strategy used to delete exon 1. PGK-hprt and MC1-tk expression cassettes are shown.

FIG. 25 Nucleotide and amino acid sequence of human O1-236 (SEQ ID NO: 16 and SEQ ID NO: 17).

### DETAILED DESCRIPTION OF THE INVENTION

The present invention provides three novel proteins, O1-180, O1-184, O1-236, the polynucleotide sequences that encode them, and fragments and derivatives thereof. Expression of O1-180, O1-184, O1-236 is highly tissue-specific, being expressed in cells primarily in ovarian tissue. In one embodiment, the invention provides a method for detection of a cell proliferative or degenerative disorder of the ovary, which is associated with expression of O1-180, O1-184 or O1-236. In another embodiment, the invention provides a method for treating a cell proliferative or degenerative disorder associated with abnormal expression of O1-180, O1-184, O1-236 by using an agent which suppresses or enhances their respective activities.

Based on the known activities of many other ovary specific proteins, it can be expected that O1-180, O1-184 and O1-236, as well as fragments and derivatives thereof, will also possess biological activities that will make them useful as diagnostic and therapeutic reagents.

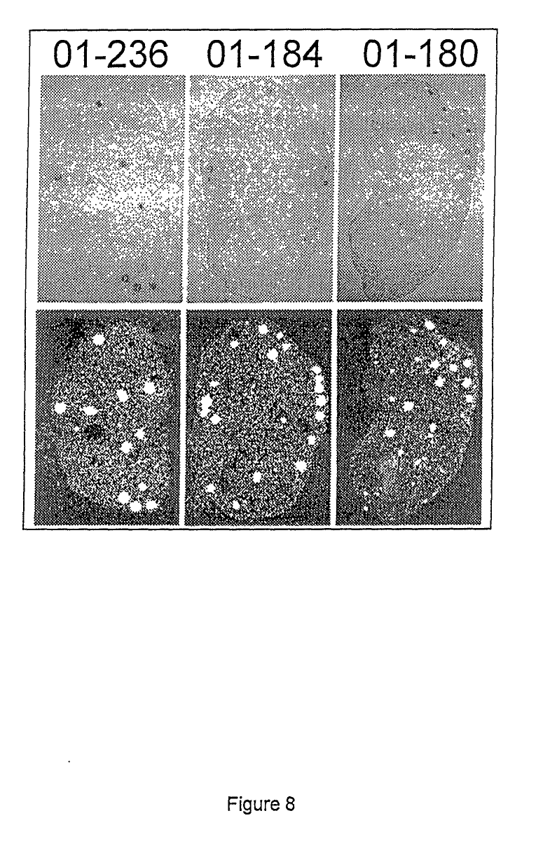

For example, GDF-9 is an oocyte-expressed gene product which has a similar pattern of expression as O1-180, O1-184, and O1-236. We have shown that mice lacking GDF-9 are infertile at a very early stage of follicular development, at the one-layer primary follicle stage (Dong, et al.). These studies demonstrate that agents which block GDF-9 function would be useful as contraceptive agents in human females. Since O1-180, O1-184, and O1-236 have an expression pattern in the oocyte (FIG. 8) which is nearly identical to GDF-9, this suggests that mice and humans or any other mammal lacking any of all of these gene products would also be infertile. Thus, blocking the function of any or all of these gene products would result in a contraceptive action.

Another regulatory protein that has been found to have ovary-specific expression is inhibin, a specific and potent polypeptide inhibitor of the pituitary secretion of FSH. Inhibin has been isolated from ovarian follicular fluid. Because of its suppression of FSH, inhibin has been advanced as a potential contraceptive in both males and females. O1-180, O1-184 and O1-236 may possess similar biological activity since they are also ovarian specific peptides. Inhibin has also been shown to be useful as a marker for certain ovarian tumors (Lappohn, et al., N. Engl. J Med., 321:790, 1989). O1-180, O1-184, O1-236 may also be useful as markers for identifying primary and metastatic neoplasms of ovarian origin. Likewise, mice which lack inhibin develop granulosa cell tumors (Matzuk et al., 1992). Similarly, O1-180, O1-184 and O1-236 may be useful as indicators of developmental anomalies in prenatal screening procedures. Mullerian inhibiting substance (MIS) peptide, which is produced by the testis and is responsible for the regression of the Mullerian ducts in the male embryo, has been shown to inhibit the growth of human ovarian cancer in nude mice (Donahoe, et al., Ann. Surg., 194:472, 1981). O1-180, O1-184 and O1-236 may function similarly and may, therefore, be targets for anti-cancer agents, such as for the treatment of ovarian cancer.

O1-180, O1-184 and O1-236, and agonists and antagonists thereof can be used to identify agents which inhibit fertility (e.g., act as a contraceptive) in a mammal (e.g., human). Additionally, O1-180, O1-184 and O1-236 and agonists and antagonists thereof can be used to identify agents which enhance fertility (e.g., increase the success

of in vivo or in vitro fertilization) in a mammal. Likewise, assays of these or related oocyte-expressed gene products can be used in diagnostic assays for detecting forms of infertility (e.g., in an assay to analyze activity of these gene products) or other diseases (e.g., germ cell tumors, polycystic ovary syndrome).

O1-180, O1-184 and O1-236 or agents which act on these pathways may also function as growth stimulatory factors and, therefore, be useful for the survival of various cell populations in vitro. In particular, if O1-180, O1-184 and/or O1-236 play a role in oocyte maturation, they may be useful targets for in vitro fertilization procedures, e.g., in enhancing the success rate.

The term “substantially pure” as used herein refers to O1-180, O1-184 and O1-236 which are substantially free of other proteins, lipids, carbohydrates or other materials with which they are naturally associated. One skilled in the art can purify O1-180, O1-184 and O1-236 using standard techniques for protein purification. The substantially pure polypeptide will yield a single major band on a non-reducing polyacrylamide gel. The purity of the O1-180, O1-184 and O1-236 polypeptides can also be determined by amino-terminal amino acid sequence analysis. O1-180, O1-184 and O1-236 polypeptides includes functional fragments of the polypeptides, as long as their activities remain. Smaller peptides containing the biological activities of O1-180,

O1-184 and O1-236 are included in the invention.

The invention provides polynucleotides encoding the O1-180, O1-184 and O1-236 proteins and fragments and derivatives thereof. These polynucleotides include DNA, cDNA and RNA sequences which encode O1-180, O1-184 or O1-236. It is understood that all polynucleotides encoding all or a portion of O1-180, O1-184 and/or O1-236 are also included herein, as long as they encode a polypeptide with the activity of O1-180, O1-184 or O1-236. Such polynucleotides include naturally occurring, synthetic, and intentionally manipulated polynucleotides. For example, polynucleotides of O1-180, O1-184 or O1-236 may be subjected to site-directed mutagenesis. The polynucleotide sequences for O1-180, O1-184 and O1-236 also includes antisense sequences. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequences of O1-180, O1-184 and O1-236 polypeptides encoded by the nucleotide sequences are functionally unchanged.

Minor modifications of the recombinant O1-180, O1-184 and O1-236 primary amino acid sequences may result in proteins which have substantially equivalent activity as compared to the respective O1-180, O1-184 and O1-236 polypeptides described herein. Such modifications may be deliberate, as by site-directed mutagenesis, or may be spontaneous. All of the polypeptides produced by these modifications are included herein as long as the biological activity of O1-180, O1-184 or O1-236 still exists. Further, deletion of one or more amino adds can also result in a modification of the structure of the resultant molecule without significantly altering its biological activity. This can lead to the development of a smaller active molecule which would have broader utility. For example, one could remove amino or carboxy terminal amino acids which may not be required for biological activity of O1-180, O1-184 or O1-236.

The nucleotide sequences encoding the O1-180, O1-184 and O1-236 polypeptides of the invention include the disclosed sequences and conservative variations thereof. The term“conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic acid for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

For the purpose of this invention, the term “derivative” shall mean any molecules which are within the skill of the ordinary practitioner to make and use, which are made

by derivatizing the subject compound, and which do not destroy the activity of the derivatized compound. Compounds which meet the foregoing criteria which diminish,

but do not destroy, the activity of the derivatized compound are considered to be within the scope of the term “derivative.” Thus, according to the invention, a derivative of a compound comprising amino acids in a sequence corresponding to the sequence of O1-180, O1-184 or O1-236, need not comprise a sequence of amino acids that corresponds exactly to the sequence of O1-180, O1-184 or O1-236, so long as it retains a measurable amount of the activity of the O1-180, O1-184 or O1-236.

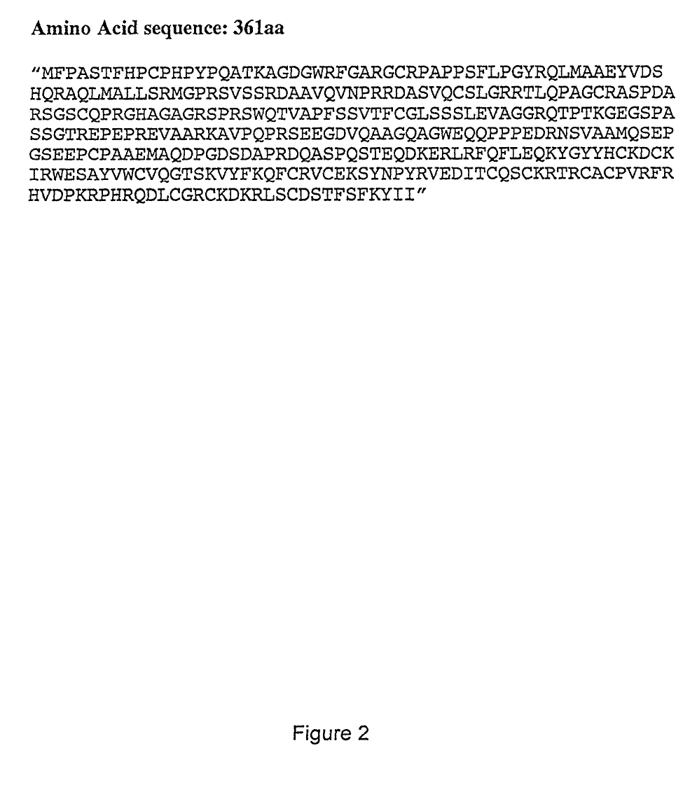

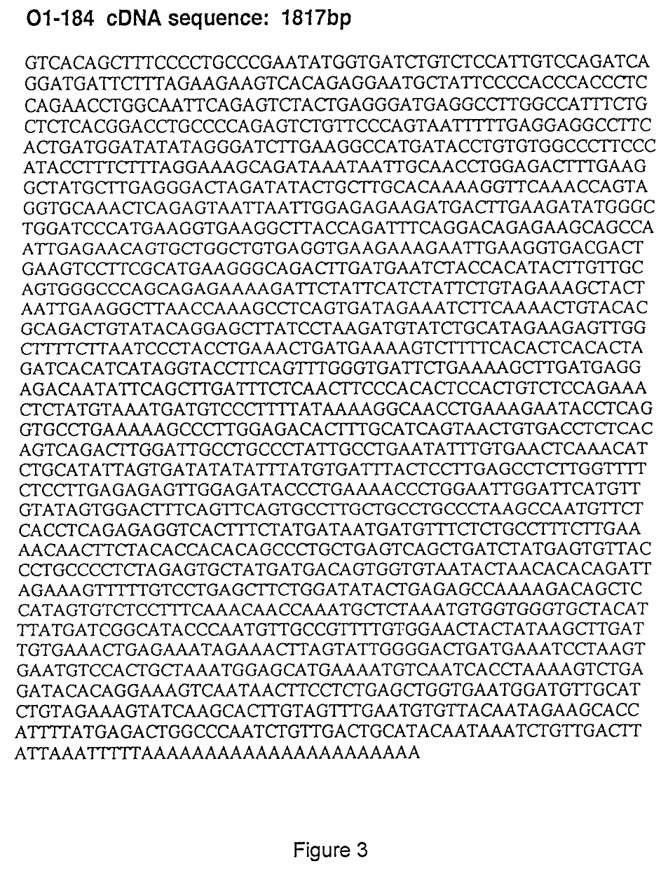

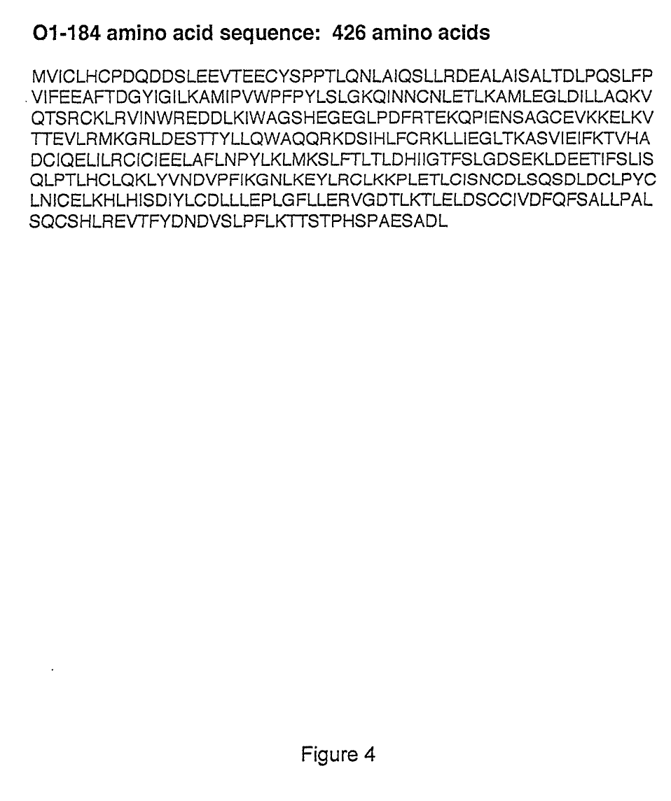

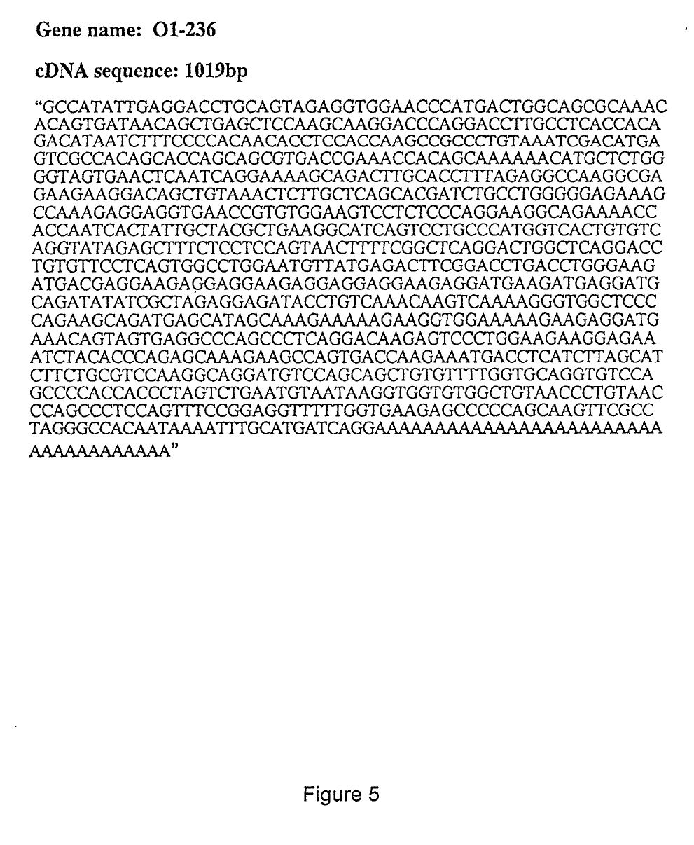

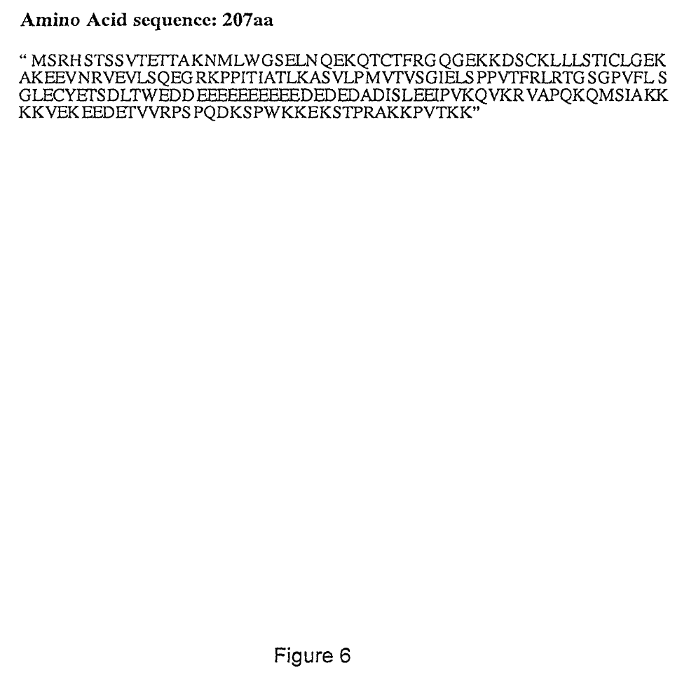

Fragments of proteins are seen to include any peptide that contains 6 contiguous amino acids or more that are identical to 6 contiguous amino acids of either of the sequences shown in FIGS. 2 (SEQ ID NO: 2), 4 (SEQ ID NO: 4), 6 (SEQ ID NO: 6), 11 and 14. Fragments that contain 7, 8, 9, 10, 11, 12, 13, 14 and 15 or more contiguous amino acids or more that are identical to a corresponding number of amino acids of any of the sequences shown in FIGS. 2 (SEQ ID NO: 2), 4 (SEQ ID NO: 4), 6 (SEQ ID NO: 6), 11 and 14 are also contemplated. Fragments may be used to generate antibodies. Particularly useful fragments will be those that make up domains of O1-180, O1-184 or O1-236. Domains are defined as portions of the proteins having a discrete tertiary structure and that is maintained in the absence of the remainder of the protein. Such structures can be found by techniques known to those skilled in the art. The protein is partially digested with a protease such as subtilisin, trypsin, chymotrypsin or the like and then subjected to polyacrylamide gel electrophoresis to separate the protein fragments. The fragments can then be transferred to a PVDF membrane and subjected to micro sequencing to determine the amino acid sequence of the N-terminal of the fragments.

DNA sequences of the invention can be obtained by several methods. For example, the DNA can be isolated using hybridization or amplification techniques which are well known in the art. These include, but are not limited to: 1) hybridization of genomic or cDNA libraries with probes to detect homologous nucleotide sequences, 2) antibody screening of expression libraries to detect cloned DNA fragments with shared structural features, or 3) use of oligonucleotides related to these sequences and the technique of the polymerase chain reaction.

Preferably the O1-180, O1-184 and O1-236 polynucleotides of the invention are derived from a mammalian organism, and most preferably from a mouse, rat, pig, cow or human. Screening procedures which rely on nucleic acid hybridization make it possible to isolate any gene sequence from any organism, provided the appropriate probe is available. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA done by the hybridization of the target DNA to that single probe in the mixture which is its complete complement (Wallace, et al., Nucl. Acid Res., 9:879, 1981).

The development of specific DNA sequences encoding O1-180, O1-184 and O1-236 can also be obtained by: 1) isolation of double-stranded DNA sequences from the genomic DNA; 2) chemical manufacture of a DNA sequence to provide the necessary codons for the polypeptides of interest; and 3) in vitro synthesis of a double stranded DNA sequence by reverse transcription of mRNA isolated from a eukaryotic donor cell. In the latter case, a double-stranded DNA complement of mRNA is eventually formed which is generally referred to as cDNA.

Of the three above-noted methods for developing specific DNA sequences for use in recombinant procedures, the isolation of genomic DNA isolates is the least common. This is especially true when it is desirable to obtain the microbial expression of mammalian polypeptides due to the presence of introns.

The synthesis of DNA sequences is frequently the method of choice when the entire sequence of amino acid residues of the desired polypeptide product is known. When the entire sequence of amino acid residues of the desired polypeptides is not known, the direct synthesis of DNA sequences is not possible and the method of choice is the synthesis of cDNA sequences. Among the standard procedures for isolating cDNA sequences of interest is the formation of plasmid- or phage-carrying cDNA libraries which are derived from reverse transcription of mRNA which is abundant in donor cells that have a high level of genetic expression. When used in combination with polymerase chain reaction technology, even rare expression products can be cloned. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay, et al., Nucl. Acid Res., 11:2325, 1983).

A cDNA expression library, such as lambda gt11, can be screened indirectly for O1-180, O1-184 and/or O1-236 peptides having at least one epitope, using antibodies specific for O1-180, O1-184 and/or O1-236. Such antibodies can be either polyclonally or monoclonally derived and used to detect expression product indicative of the presence of O1-180, O1-184 and/or O1-236 cDNA.

DNA sequences encoding O1-180, O1-184 or O1-236 can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

In the present invention, the O1-180, O1-184 and/or O1-236 polynucleotide sequences may be inserted into a recombinant expression vector. The term “recombinant expression vectors” refers to a plasmid, virus or other vehicle known in the art that has been manipulated by insertion or incorporation of the O1-180, O1-184 or O1-236 genetic sequences. Such expression vectors contain a promoter sequence which facilitates the efficient transcription of the inserted genetic sequence of the host. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg, et al.,Gene, 56:125, 1987), the pMSXND expression vector for expression in mammalian cells (Lee and Nathans, J. Biol. Chem., 263:3521, 1988) and baculovirus-derived vectors for expression in insect cells. The DNA segment can be present in the vector operably linked to regulatory elements, for example, a promoter (e.g., T7, metallothionein 1, or polyhedrin promoters). Polynucleotide sequences encoding O1-180, O1-184 or O1-236 can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

Transformation of a host cell with recombinant DNA may be carried out by conventional techniques as are well known to those skilled in the art. Where the host is prokaryotic, such as E coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be co-transformed with DNA sequences encoding the O1-180, O1-184 or O1-236 cDNA sequences of the invention, and a second foreign DNA molecule encoding a selectable phenotype, such as the neomycin resistance gene. Another method is to use a eukaryotic viral vector, such as simian virus 40 (SV40) or bovine papilloma virus, to transiently infect or transform eukaryotic cells and express the protein. (see for example, Eukaryotic Viral Vectors, Cold Spring Harbor Laboratory, Gluzman ed., 1982).

Isolation and purification of microbial expressed polypeptide, or fragments thereof, provided by the invention, may be carried out by conventional means induding preparative chromatography and immunological separations involving monoclonal or polyclonal antibodies.

The invention includes antibodies immunoreactive with O1-180, O1-184 or O1-236 polypeptides or functional fragments thereof. Antibody which consists essentially of pooled monoclonal antibodies with different epitopic specificities, as well as distinct monoclonal antibody preparatory are provided. Monoclonal antibodies are made from antigen containing fragments of the protein by methods well known to those skilled in the art (Kohler, et al., Nature, 256:495, 1975). The term antibody as used in this invention is meant to include intact molecules as well as fragments thereof, such as Fab and F(ab′)2, which are capable of binding an epitopic determinant on O1-180, O1-184 or O1-236.

The term “cell-proliferative disorder” denotes malignant as well as non-malignant cell populations which often appear to differ from the surrounding tissue both morphologically and genotypically. The O1-180, O1-184 and O1-236 polynucleotides that are antisense molecules are useful in treating malignancies of the various organ systems, particularly, for example, the ovaries. Essentially, any disorder which is etiologically linked to altered expression of O1-180, O1-184 or O1-236 could be considered susceptible to treatment with a O1-180, O1-184 or O1-236 suppressing reagent, respectively.

The invention provides a method for detecting a cell proliferative disorder of the ovary which comprises contacting an anti-O1-180, O1-184 or O1-236 antibody with a cell suspected of having an O1-180, O1-184 or O1-236 associated disorder and detecting binding to the antibody. The antibody reactive with O1-180, O1-184 or O1-236 is labeled with a compound which allows detection of binding to O1-180, O1-184 or O1-236, respectively. For purposes of the invention, an antibody specific for an O1-180, O1-184 or O1-236 polypeptide may be used to detect the level of O1-180, O1-184 or O1-236, respectively, in biological fluids and tissues. Any specimen containing a detectable amount of antigen can be used. A preferred sample of this invention is tissue of ovarian origin, specifically tissue containing oocytes or ovarian follicular fluid. The level of O1-180, O1-184 or O1-236 in the suspect cell can be compared with the level in a normal cell to determine whether the subject has an O1-180, O1-184 or O1-236-associated cell proliferative disorder. Preferably the subject is human. The antibodies of the invention can be used in any subject in which it is desirable to administer in vitro or in vivo immunodiagnosis or immunotherapy. The antibodies of the invention are suited for use, for example, in immuno assays in which they can be utilized in liquid phase or bound to a solid phase carrier. In addition, the antibodies in these immunoassays can be detectably labeled in various ways. Examples of types of immunoassays which can utilize antibodies of the invention are competitive and noncompetitive immunoassays in either a direct or indirect format. Examples of such immunoassays are the radioimmunoassay (RIA) and the sandwich (ELISA) assay. Detection of the antigens using the antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The term “cell-degenerative disorder” denotes the loss of any type of cell in the ovary, either directly or indirectly. For example, in the absence of GDF-9, there is a block in the growth of the granulosa cells leading to eventual degeneration (i.e., death) of the oocytes (Dong et al., 1996). This death of the oocyte appears to lead to differentiation of the granulosa cells. In addition, in the absence of GDF-9, no normal thecal cell layer is formed around the follicles. Thus, in the absence of one oocyte-specific protein, GDF-9, there are defects in three different cell lineages, oocytes, granulosa cells, and thecal cells. In a similar way, death or differentiation of these various cell lineages could be affected by absence or misexpression of O1-180, O1-184, or O1-236. Furthermore, absence or misexpression of O1-180, O1-184, or O1-236 could result in defects in the oocyte/egg leading to the inability of the egg to be fertilized by spermatozoa.

The antibodies of the invention can be bound to many different carriers and used to detect the presence of an antigen comprising the polypeptide of the invention. Samples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the antibody, or will be able to ascertain such, using routine experimentation.

Another technique which may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, puridoxal, and fluorescein, which can react with specific anti-hapten antibodies.

In using the monoclonal antibodies of the invention for the in vivo detection of antigen, the detectably labeled antibody is given a dose which is diagnostically effective. The term “diagnostically effective” means that the amount of detectably labeled monoclonal antibody is administered in sufficient quantity to enable detection of the site having the antigen composing a polypeptide of the invention for which the monoclonal antibodies are specific. The concentration of detectably labeled monoclonal antibody which is administered should be sufficient such that the binding to those cells having the polypeptide is detectable compared to the background. Further, it is desirable that the detectably labeled monoclonal antibody be rapidly cleared from the circulatory system in order to give the best target-to-background signal ratio. As a rule, the dosage of detectably labeled monoclonal antibody for in vivo diagnosis will vary depending on such factors as age, sex, and extent of disease of the individual. Such dosages may vary, for example, depending on whether multiple injections are given, antigenic burden, and other factors known to those of skill in the art.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that deleterious radiation with respect to the host is minimized. Ideally, a radioisotope used for in vivo imaging will lack a particle emission, but produce a large number of photons in the 140-250 keV range, which may readily be detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA)

and similar molecules. Typical examples of metallic ions which can be bound to the monoclonal antibodies of the invention are ¹¹¹In, ⁹⁷Ru, ⁶⁷Ga, ⁶⁸Ga, ⁷²As, ⁸⁹Zr and 201Ti.

The monoclonal antibodies of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes

are used for camera imaging and paramagnetic isotopes for MRI. Elements which are particularly useful in such techniques include ¹⁵⁷Gd, ⁵⁵Mn, ¹⁶²Dy, ⁵⁵Cr and ⁵⁶Fe.

The monoclonal antibodies of the invention can be used in vitro and in vivo to monitor the course of amelioration of an O1-180, O1-184 or O1-236-associated disease in a subject. Thus, for example, by measuring the increase or decrease in the number of cells expressing antigen comprising a polypeptide of the invention or changes in the concentration of such antigen present in various body fluids, it would be possible to determine whether a particular therapeutic regimen aimed at ameliorating the O1-180, O1-184 or O1-236-associated disease is effective. The term “ameliorate” denotes a lessening of the detrimental effect of the O1-180, O1-184 or O1-236-associated disease in the subject receiving therapy.

The present invention identifies nucleotide sequences that can be expressed in an altered manner as compared to expression in a normal cell, therefore, it is possible to design appropriate therapeutic or diagnostic techniques directed to this sequence. Thus, where a cell-proliferative disorder is associated with the expression of O1-180, O1-184 or O1-236, nucleic acid sequences that interfere with the expression of O1-180, O1-184 or O1-236, respectively, at the translational level can be used. This approach utilizes, for example, antisense nucleic acids or ribozymes to block translation of a specific O1-180, O1-184 or O1-236 mRNA, either by masking that mRNA with an antisense nucleic acid or by cleaving it with a ribozyme.

Antisense nucleic acids are DNA or RNA molecules that are complementary to at least a portion of a specific mRNA molecule (Weintraub, Scientific American, 262:40, 1990). In the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target O1-180, O1-184 or O1-236-producing cell. The use of antisense methods to inhibit the in vitro translation of genes is well known in the art (Marcus-Sakura, Anal.Biochem., 172:289, 1988).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, J.Amer.Med. Assn., 260:3030, 1988). A major advantage of this approach is that, because they are sequence-specific, only mRNAs with particular sequences are inactivated.

There are two basic types of ribozymes namely, tetrahymena-type (Hasselhoff, Nature, 334:585, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-based recognition sequences are preferable to shorter recognition sequences.

The present invention also provides gene therapy for the treatment of cell proliferative or degenerative disorders which are mediated by O1-180, O1-184 or O1-236 proteins. Such therapy would achieve its therapeutic effect by introduction of the respective O1-180, O1-184 or O1-236 cDNAs or O1-180, O1-184, or O1-236 antisense polynucleotide into cells having the proliferative or degenerative disorder. Delivery of O1-180, O1-184, or O1-236 cDNAs or antisense O1-180, O1-184 or O1-236 polynucleotides can be achieved using a recombinant expression vector such as a chimeric virus or a colloidal dispersion system.

Especially preferred for therapeutic delivery of cDNAs or antisense sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting an O1-180, O1-184 or O1-236 sequence of interest into the viral vector, along with another gene which encodes the ligand for a receptor on a specific target cell, for example, the vector is now target specific. Retroviral vectors can be made target specific by inserting, for example, a polynucleotide encoding a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing a O1-180, O1-184 or O1-236 cDNA or O1-180, O1-184, or O1-236 antisense polynucleotides.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packing mechanism to recognize an RNA transcript for encapsidation. Helper cell lines which ave deletions of the packaging signal include, but are not limited to ψ2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for O1-180, O1-184 or O1-236 cDNAs or O1-180, O1-184, or O1-236 antisense polynucleotides is a colloidal dispersion system. Colloidal dispersion systems include macromolecule complexes, nanocapsules complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high exigency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Manning, et al., Biotechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

Due to the expression of O1-180, O1-184 and O1-236 in the reproductive tract, there are a variety of applications using the polypeptides, polynucleotides and antibodies of the invention, related to contraception, fertility and pregnancy. O1-180, O1-184 and O1-236 could play a role in regulation of the menstrual cycle and, therefore, could be useful in various contraceptive regimens.

The following examples are intended to illustrate but not limit the invention. While they are typical of those that might be used, other procedures known to those skilled in the art may alternatively be used.

### EXAMPLE 1

### Creation of a cDNA Subtractive Hybridization Library

Ovaries from GDF-9-deficient mice are histologically very different from wild-type ovaries due to the early block in folliculogenesis. In particular, one layer primary follicles are relatively enriched in GDF-9-deficient ovaries and abnormal follicular nests are formed after oocyte loss. We took advantage of these differences in ovary composition and related them to alterations in gene expression patterns to clone novel ovary-expressed transcripts which are upregulated in the GDF-9-deficient ovaries.

Ovaries from either GDF-9-deficient mice (C57BL/6/129SvEv hybrid) or wild-type mice were collected and polyA+ mRNA was made from each pool. Using a modified version of the CLONTECH PCR-Select Subtraction kit, we generated a pBluescript SK+plasmid-based cDNA library which was expected to be enriched for sequences upregulated in the GDF-9-deficient ovaries. Ligations into the NotI site of pBluescript

SK+ were performed with a low molar ratio of EagI-digested cDNA fragment inserts to vector to prevent multiple inserts into the vector. Transformations were performed, and >1000 independent bacterial clones were picked and stored in glycerol at −80° C. The remainder of the ligation mix was stored at −80° C. for future transformations.

### EXAMPLE 2

### Initial Sequence Analysis of pOvaryl (pO1) Library Inserts

We performed sequence analysis of 331 inserts from the pO1 subtractive hybridization of cDNA library. An Applied Biosystems 373 DNA Sequencer was used to sequence these clones. BLAST searches were performed using the National Center for Biotechnology Information databases. Novel sequences were analyzed for open reading frames and compared to previously identified novel sequences using DNASTAR analysis programs. A summary of the data is presented in Table 1. As shown, the majority of the clones were known genes or match mouse or human ESTs. 9.4% of the clones fail to match any known sequence in the database.

### EXAMPLE 3

### Expression Analysis and cDNA Screening of Ovarian-expressed Genes with No Known Function

The functions of the pO1-library gene products which match ESTs or where there is no match are not known (Table 1). Northern blot analysis was performed on all cDNAs which failed to match sequences in any database. Additionally, sequences matching ESTs derived predominantly from mouse 2-cell embryo cDNA libraries (e.g., O1-91, O1-184, and O1-236) were analyzed. The rationale for analyzing this last group of ESTs is that mRNAs expressed at high levels in oocytes may persist until the 2-cell stage and may play a role in early embryonic development including fertilization of the egg or fusion of the male and female pronuclei.

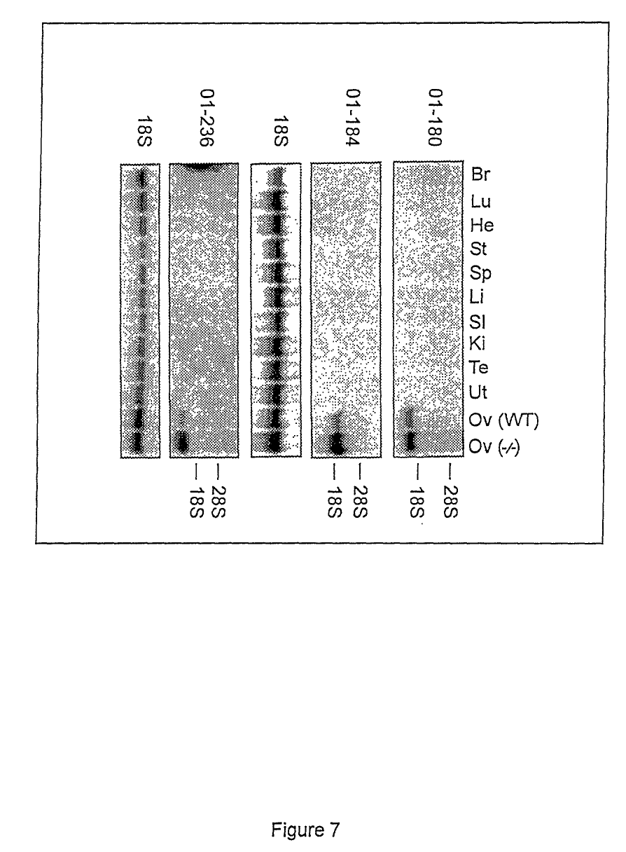

The results of the initial screen of novel ovarian genes is presented in Table 2. Northern blot analysis of 23 clones demonstrated that 8 of these clones were upregulated in the GDF-9-deficient ovary indicating the subtractive hybridization protocol used was adequate. Northern blot analysis using total RNA isolated from either adult C57BL/6/129SvEv hybrid mice (the ovarian RNA) or Swiss WEBSTER mice (all other tissues) also demonstrated that four of these clones including 2 clones which matched ESTs sequenced from 2-cell libraries were only expressed in the ovary (FIG. 7). The O1-236 fragment probe (749 bp) detected a transcript of approximately 1.0 kb (FIG. 7). Several clones have so far been analyzed for their ovarian localization by in situ hybridization analysis (FIG. 8). Clones O1-180, O1-184, and O1-236 were oocyte-specific and expressed in oocytes of primary (one-layer) preantral follicles through ovulation (FIG. 8).

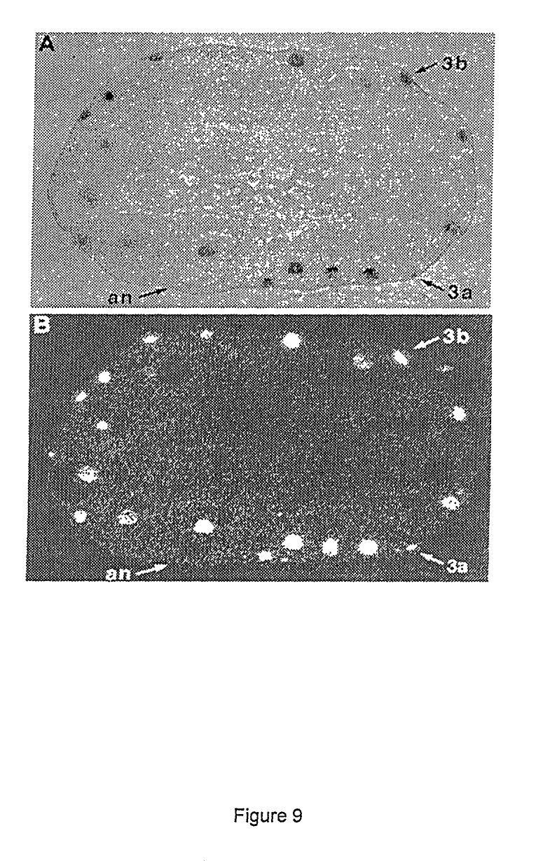

The O1-236 gene product is oocyte-specific (FIG. 9). O1-236 is not expressed in oocytes of primordial (type 2) or small type 3a follicles (Pedersen et al., Journal of Reproduction and Fertility, 17:555-557, 1968) (data not shown) but is first detected in oocytes of intermediate-size type 3a follicles and all type 3b follicles (i.e., follicles with >20 granulosa cells surrounding the oocyte in largest cross-section). Expression of the O1-236 mRNA persisted through the antral follicle stage. Interestingly, the oocyte-specific expression pattern of the O1-236 gene product parallels the expression of other oocyte-specific genes which we have studied including Gdf9 (McGrath et al., Molecular Endocrinology 9:131-136 (1995)) and bone morphogenetic protein 15 (Dube et al., Molecular Endocrinology 12:1809-1817, 1998).

### EXAMPLE 4

### Cloning of Ovary Specific Genes, Including Mouse Npm2, the Mammalian Ortholog of Xenopus laevis Nucleoplasmin (Xnpm2)

Wild-type ovary and GDF-9-deficient ZAP Express ovary cDNA libraries were synthesized and were screened to isolate full-length cDNAs for the above-mentioned three clones. Each fill-length cDNA was again subjected to database searches and analyzed for an open reading frame, initiation ATG, and protein homology. The full-length cDNAs approximate the mRNA sizes determined from Northern blot analysis. Database searches using the predicted amino acid sequence permitted the identification of important domains (e.g., signal peptide sequences, transmembrane domains, zinc fingers, etc.) which will be useful to define the possible function and cellular localization of the novel protein.

The O1-236 partial cDNA fragment identified in Example 1 was used to screen Matzuk laboratory ZAP Express (Stratagene) ovarian cDNA libraries generated from either wild-type or GDF-9 deficient ovaries as per manufacturer's instructions and as described previously (Dube, et al., Molecular Endocrinology, 12:1809-1817 (1998)). In brief, approximately 300,000 clones of either wild-type or GDF-9 knockout mouse ovary cDNA libraries were hybridized to [α-³²P] dCTP random-primed probes in Church's solution at 63° C. Filters were washed with 0.1× Church's solution and exposed overnight at −80° C.

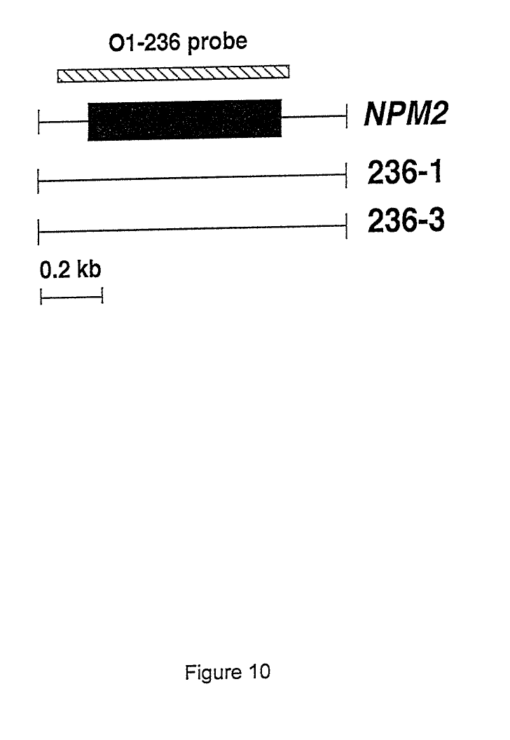

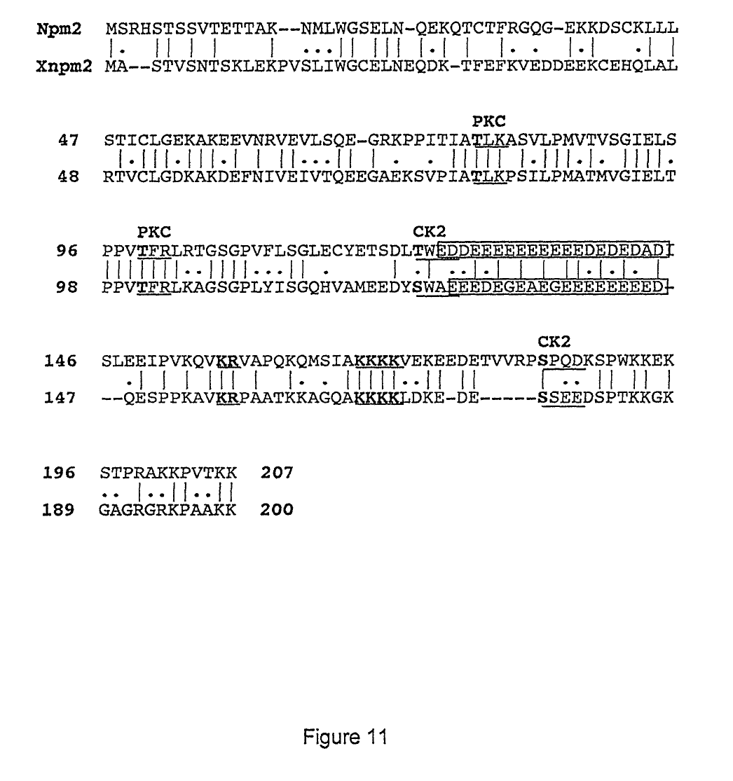

Upon primary screening of the mouse ovarian cDNA libraries, the O1-236 cDNA fragment detected 22 positive phage clones out of 300,000 screened. Two of these clones (236-1 and 236-3), which approximated the mRNA size and which were derived from the two independent libraries, were analyzed further by restriction endonuclease digestion and DNA sequence analysis. These independent clones form a 984 bp overlapping contig (excluding the polyA sequences) and encode a 207 amino acid open reading frame (FIG. 10). Including the polyA tail, this sequence approximates the 1.0 kb mRNA seen by Northern blot analysis suggesting that nearly all of the 5′ UTR sequence has been isolated. When the nucleotide sequence is subjected to public database search, no significant matches were derived. However, database search with the 207 amino acid open reading frame demonstrated high homology with several nucleoplasmin homologs from several species. Interestingly, O1-236 shows highest homology with Xenopus laevis nucleoplasmin. At the amino acid level, O1-236 is 48% identical and 71% similar to Xenopus laevis nucleoplasmin (FIG. 11). Based on this homology and the expression patterns of both gene products in oocytes, we have termed our gene Npm2 since it is the mammalian ortholog of Xenopus laevis nucleoplasmin [called Xnpm2 in (MacArthur et al., Genomics rs:137-140 (1997))]

When the Npm2 and nucleoplasmin sequences are compared, several interesting features are realized. Nucleoplasmin has a bipartite nuclear localization signal consisting of KR-(X)₁₀⁻ KKKK (Dingwall, et al. EMBO J 6:69-74 (1987)). Deletion of either of these basic amino acid clusters in nucleoplasmin prevents translocation to the nucleus (Robbins et al. Cell 64:615-623) (1991)). When the Npm2 sequence is analyzed, this bipartite sequence is 100% conserved between the two proteins (FIG. 11). Thus, Npm2 would be predicated to translocate to the nucleus where it would primarily function.

Also conserved between Npm2 and nucleplasmin is a long stretch of negatively charged residues. Amino acids 125-144 of Npm2 and amino acids 128-146 of nucleoplasmin are mostly glutamic acid and aspartic acid residues, with 19 out of the 20 residues for Npm2 and 16 out of the 19 residues for nucleoplasmin either Asp or Glu. This region of Xenopus laevis nucleoplasmin has been implicated to bind the positively charged protamines and histones. Thus, a similar function for this acidic region of Npm2 is predicted.

The last obvious feature of the Npm2 and nucleoplasmin sequences is the high number of serine and threonine residues. The Npm2 sequence contains 19 serine and 17 threonines (i.e., 17.2% of the residues) and nucleoplasmin has 12 serine and 11 threonine residues (i.e., 11.5% of the residues). Multiple putative phosphorylation sites are predicted from the Npm2 and nucleoplasmin sequences. Several putative phosphorylation sequences that are conserved between the two proteins are shown in FIG. 11. Phosphorylation of nucleoplasmin is believed to increase its translocation to the nucleus and also its activity (Sealy et al. Biochemistry 25: 3064-3072 (1986); Cotten et al. Biochemistry 25:5063-5069 (1986); Vancurova et al. J Cell Sci 108:779-787 (1995); Leno et al. J Biol Chem 271: 7253-7256 (1996)). Similarly, phosphorylation may also alter Npm2 activity. Thus, since both Npm2 and Xenopus laevis nucleoplasm are oocyte (and egg)-specific at the mRNA level and share highest identity, we conclude that Npm2 and nucleoplasmin are orthologs.

### EXAMPLE 5

### Structure of the Npm2 Gene

Our studies show that all three of the novel oocyte-specific cDNAs have open reading frames. As discussed above, O1-236 is the homolog of Xenopus laevis nucleoplasmin expressed exclusively in eggs.

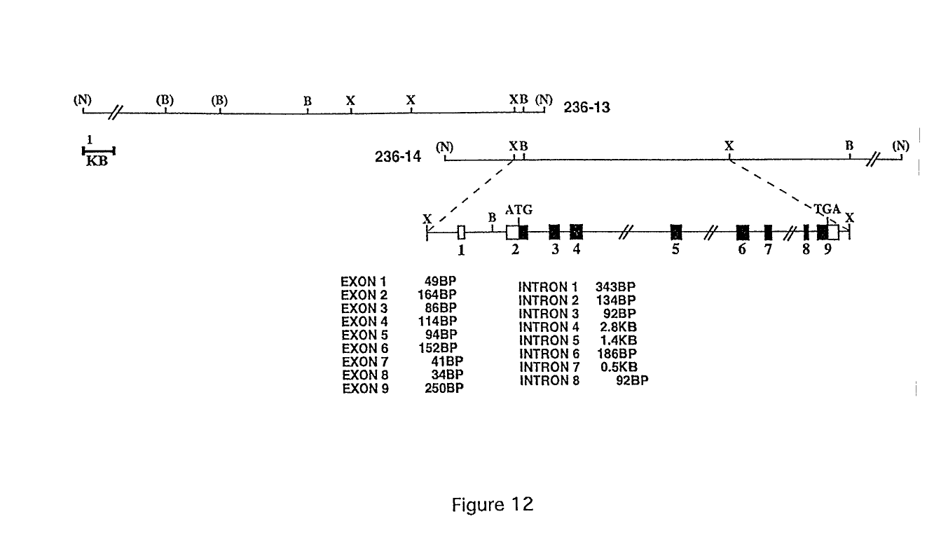

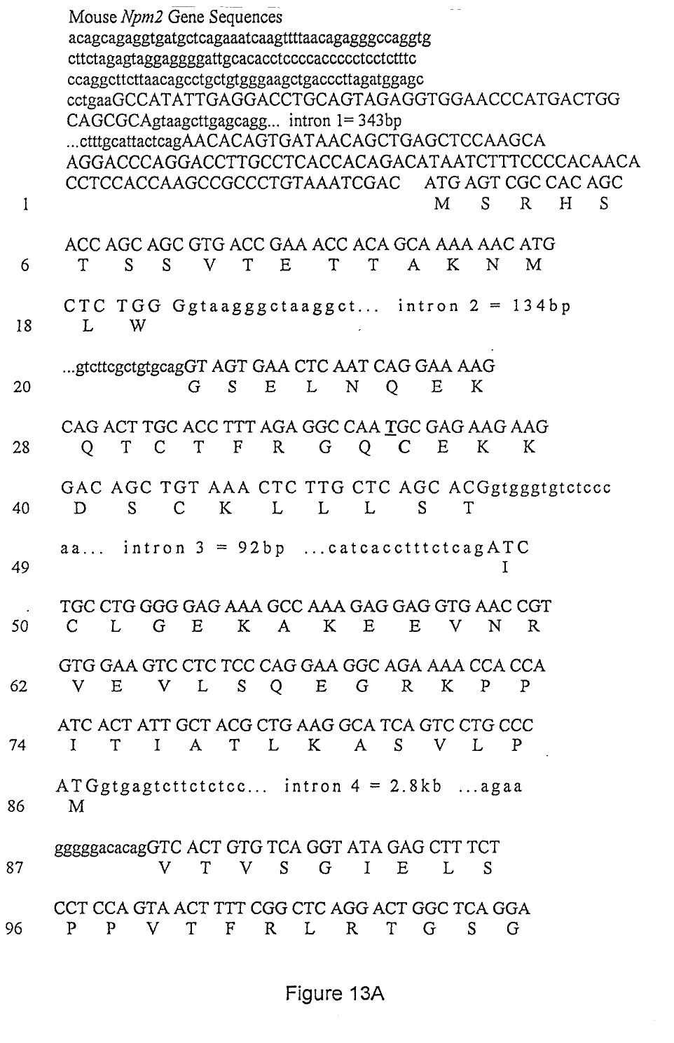

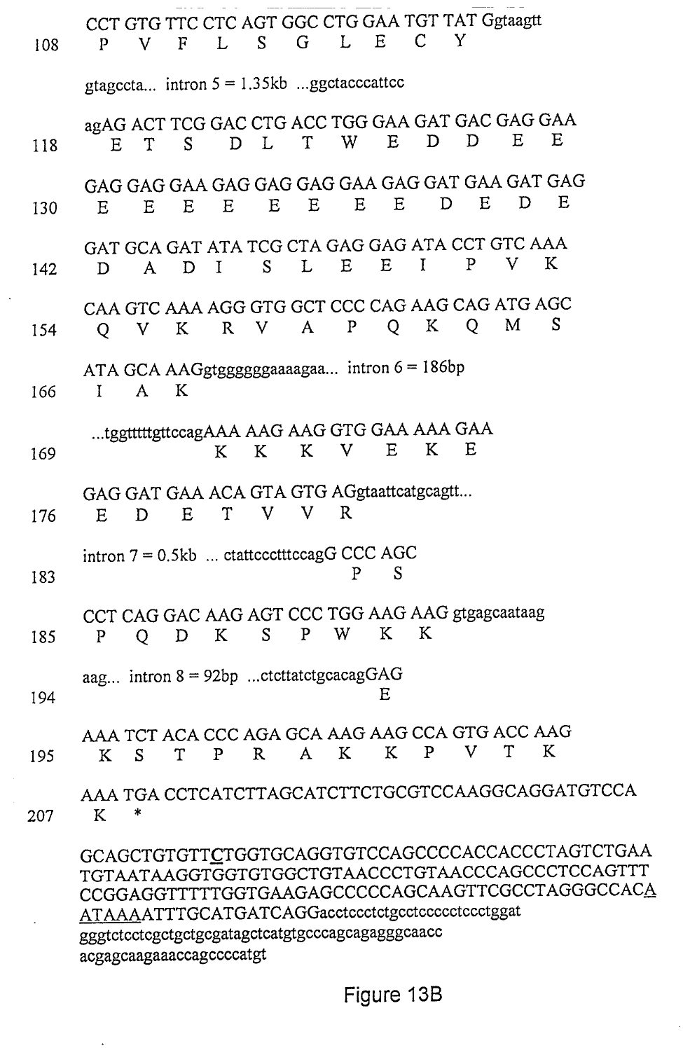

One of the full length Npm2 cDNAs (clone 236-1) was used to screen a mouse 129SvEv genomic library (Stratagene) to identify the mouse Npm2 gene. 500,000 phage were screened and 12 positive were identified. Two of these overlapping phage clones, 236-13 and 236-14 (˜37 kb of total genomic sequence), were used to determine the structure of the mouse Npm2 gene. The mouse Npm2 is encoded by 9 exons and spans ˜6.6 kb (FIGS. 12 and 13A and 13B (SEQ ID NO: 7-14)). Two moderate size introns (introns 4 and 5) contribute the majority of the gene size. The initiation ATG codon resides in exon 2 and the termination codon in exon 9. The splice donor and acceptor sites (FIGS. 13A and 13B (SEQ ID NO: 7-14)) match well with the consensus sequences found in rodents, and all of the intron-exon boundaries conform to the “GT-AG” rule (Senapathy et al. Methods Enzymol 183:252-278 (1990)). A consensus polyadenylation signal sequence (AATAAA) is found upstream of the polyA tracts which are present in the two isolated cDNAs (FIGS. 13A and 13B (SEQ ID NO: 7-14)).

Analysis of the open reading frames of O1-180 and O1-184, fails to demonstrate any structural motifs reminiscent of known proteins, suggesting that they will be functionally unique. As with O1-236, a λFixII genomic library generated from mouse strain 129SvEv will be used for the isolation of the O1-180 and O1-184 genes. Restriction enzyme digestions, Southern blot analysis, subcloning and sequence analysis will be used to determine the genomic structure including the location and sequence of exons, exon-intron boundaries, and 5′ and 3′ non-translated regions. This gene structure information will be critical in generating a gene targeting vector as described below. In addition to O1-236, we have cloned 14 mouse genes from this genomic library and aided in the analysis of another 8 genes from this library. Thus, based on our previous experience, the cloning of these mouse genes will be fairly straightforward.

### EXAMPLE 6

### Chromosomal Mapping of the Mouse Npm2 Gene

Chromosomal mapping of genes in the mouse can identify candidate genes associated with spontaneous or induced mouse mutations. For example, mapping of the TGF-β family member, growth differentiation factor-5 (GDF-5), showed that it mapped to the same chromosomal location as the gene causing brachypodism in mice. Later studies showed that mutations in GDF-5 cause autosomal dominant brachydactyly type C and two types of recessive chondrodysplasia in humans. To further aid in our functional analysis of the isolated novel ovary-specific cDNAs we are mapping these mouse genes using the Research Genetics Radiation Hybrid Panel. We have mapped several other genes in our laboratory, including O1-186 (Table 3) and therefore we believe that these studies will be fairly straightforward. This information may direct us to known mutations in the mouse mapping to the same chromosomal region associated with reproductive defects. Identification of the syntenic region on the human chromosome may identify one or more of these novel ovarian genes as candidate genes for known human diseases which map to these regions.

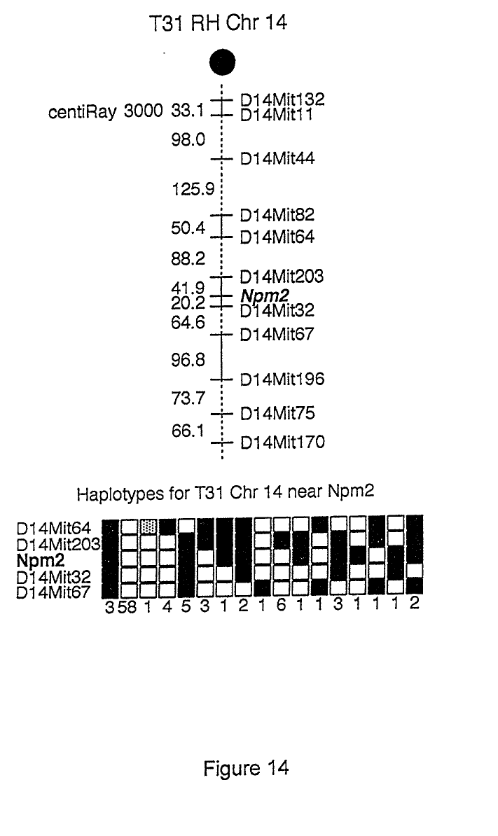

To map the mouse Npm2 gene, we used the Research Genetics radiation hybrid panel, The Jackson Laboratory Backcross DNA Panel Mapping Resource, and The Jackson Laboratory Mouse Radiation Hybrid Database. Forward (GCAAAGAAGC CAGTGACCAA GAAATGA) and reverse (CCTGATCATG CAAATTTTAT TGTGGCC) primers within the last exon were used to PCR amplify a 229 bp fragment from mouse but not hamster. Using these primers, the mouse Npm2 gene was mapped to the middle of chromosome 14 (FIG. 14). Npm2 shows linkage to D14Mit32 with a LOD of 11.2 and also has a LOD of 7.8 to D14Mit203. This region is syntenic with human chromosome 8p21.

These studies will be part of our initial efforts to identify novel gene products which may be potential targets for contraceptives or treatment of infertility in human females. As mentioned above, we have created several mouse models with defects in the ovary. We will also use ovaries from these various models (especially the GDF-9-deficient and FSH-deficient mice) to further study by in situ hybridization any ovary-specific genes. Thus, these additional studies may help to further define the factors which regulate their expression and the roles of these ovary-specific genes in vivo.

### EXAMPLE 7

### Generation of Knockout Mice Lacking Novel Ovary-expressed Genes

We will initiate studies to generate knockout mice lacking ovary-specific genes. Using the gene sequences obtained above, we will generate a targeting vector to mutate the O1-180, O1-184 and O1-236 genes in embryonic stem (ES) cells. These targeting vectors will be electroporated into the hprt-negative AB2.1 ES cell line and selected in HAT and FIAU. Clones will be processed for Southern blot analysis and screened using 5′ and 3′ external probes. ES cells with the correct mutation will be injected into blastocysts to generate chimeras and eventually heterozygotes and homozygotes for the mutant O1-180, O1-184 and O1-236 genes. Based on our success rate of transmission of mutant ES cell lines (28 independent mutant alleles from multiple ES cell lines) we do not anticipate any difficulties in generating heterozygotes and homozygotes for the mutant O1-180, O1-184 and O1-236 alleles.

Since expression of O1-180, O1-184 and O1-236 is limited to the ovary, we anticipate that these O1-180-deficient, O1-184-deficient and O1-236-deficient mice will be viable, but that females lacking these gene products will have fertility alterations (i.e., be infertile, subfertile, or superfertile). Mutant mice will be analyzed for morphological, histological and biochemical defects similar to studies we have performed in the past. These are well within the ability of the person of ordinary skill to carry out, without undue experimentation and are expected to confirm that O1-180, O1-184 and O1-236 are key intraovarian proteins required for folliculogenesis, oogenesis, or fertilization, and that in the absence of these proteins, female mice will have increased or decreased fertility. These studies will lead us to search for human reproductive conditions with similar idiopathic phenotypes.

While this invention has been particularly shown and described with references to preferred embodiment thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically therein. Such equivalents are intended to be encompassed in the scope of the claims.

### EXAMPLE 8

### Materials and Methods for Npm2

Creation and Analysis of a cDNA Subtractive Hybridization Library

Ovaries from either GDF-9-deficient mice (C57BL/6/129SvEv hybrid) or wild-type mice were collected and polyA⁺ mRNA was made from each pool. A modified version of the PCR-Select Subtraction kit (CLONTECH) was used to generate a pBluescript SK+ (Stratagene) plasmid-based cDNA library. Ligations into the NotI site of pBluescript SK+ were performed with a low molar ratio of EagI-digested cDNA fragment inserts to vector to prevent multiple inserts into the vector.

The clones from this ovary (pO1) subtractive hybridization cDNA library were sequenced using an Applied Biosystems 373 DNA Sequencer. BLAST searches were performed using the National Center for Biotechnology Information databases. The interesting partial cDNA fragments identified in the above-mentioned screen were used to screen Matzuk laboratory ZAP Express (Stratagene) ovarian cDNA libraries generated from either wild-type or GDF-9-deficient ovaries as per manufacturer's instructions and as described previously (Dube et al., 1998). In brief, approximately 300,000 clones of either wild-type or GDF-9 knockout mouse ovary cDNA libraries were hybridized to [α³²P] dCTP random-primed probes in Church's solution at 63° C. Filters were washed with 0.1× Church's solution and exposed overnight at −80° C.

Northern Blot Analysis and in situ Hybridization

Total RNA was extracted from multiple tissues of wild-type (C57B16/129SvEv) and GDF-9 knockout mice using RNA STAT-60 (Leedo Medical Laboratories, Houston, Tex.) as described by the manufacturer. 12 ug total RNA was electrophoresed on a 1.2% agarose/7.6% formaldehyde gel and transferred to Hybond-N (Amersham, Arlington Heights, Ill.) nylon membrane. The O1-236 fragment was used as the probe. The membrane was hybridized, washed, and subject to autoradiography as previously described (Mahmoudi and Lin, 1989). An 18S ribosomal RNA cDNA probe was used for the loading control.

In situ hybridization was performed as described previously (Albrecht et al., 1997; Elvin et al., 1999). Briefly, ovaries were dissected from C57B16/129SvEv mice and fixed overnight in 4% paraformaldehyde in PBS before processing, embedding in paraffin and sectioning at 5 um. The fragment O1-236 was used as the template for generating sense and antisense strands with [α³²P]-dUTP using the Riboprobe T7/SP6 combination system (Promega). Hybridization was carried out at 50-55° C. with 5×10⁶ cpm for each riboprobe per slide for 16 hours in 50% deionized formamide/0.3 M NaCl/20 mM Tris-HCl (pH 8.0)/5 mM EDTA/10 mM NaPO₄ (pH8.0)/10% dextran sulphate/1× Denhardts/0.5 mg/ml yeast RNA. High stringency washes were carried out in 2× SSC/50% formamide and 0.1× SSC at 65° C. Dehydrated sections were dipped in NTB-2 emulsion (Eastman Kodak, Rochester, N.Y.) and exposed for 4-7 days at 4° C. After the slides were developed and fixed, they were stained with hematoxylin and mounted for photography.

Preparation of Anti-Npm2 Antibodies

The cDNA encoding the full-length mouse Npm2 protein was amplified by PCR to introduce a BamH1 site before the start codon and a XhoI site before the stop codon. This PCR fragment was cloned into pET-23b(+)(Novagen) to produce a His-tagged Npm2 protein and sequenced to confirm the absence of mutations. The recombinant Npm2 protein was purified as described in the pET System Manual (Novagen). Two goats were immunized with the purified His-tagged Npm2 to produce specific and high affinity antibodies.

Immunohistochemistry

Ovaries were fixed in 4% paraformaldehyde in PBS for 2 h, processed, embedded in paraffin, and sectioned at 5 um thickness. Goat anti-Npm2 polyclonal antiserum was diluted 1:2000 in Common Antibody Dilute (BioGenex). The pre-immune goat serum from the same goat was used as a control. All section were blocked for 10 min in Universal Blocking Reagent (BioGenex), and incubated with the primary antibody for 1 h at room temperature. Npm2 detection was accomplished using anti-goat biotinylated secondary antibody, streptavidin-conjugated alkaline phosphatase label and New Fuschin substrate (BioGenex Laboratories, Inc., San Ramon, Calif.).

One to eight-cell embryos and blastocysts were fixed in 4% paraformaldehyde in PBS for 2 h in 96-well round bottom plate, washed with 0.85% saline, and embedded in a few drops of 1.5% agarose. The agarose-containing embryos were dehydrated, embedded in paraffin, and analyzed as described above.

Construction of the Npm2 Targeting Vector and Generation of Npm2-deficient Mice

A targeting vector for Npm2 was constructed to delete exons 2 and 3 and the splice junction of exon 4. The deletion targeting vector contains from left to right, 2.2 kb of 5′ Npm2 homology, a PGK-hprt expression cassette, 4.6 kb of 3′Npm2 homology and an MC1-tk (thymidine kinase) expression cassette. The linearized Npm2 targeting vector was electroporated into AB2.1 ES cells. ES cell clones were selected in M15 medium containing HAT (hypoxanthine, aminopterine and thymidine and FIAU [1-(2′-deoxy-2′-fluoro-B-D-arabinofuranosyl)-5′-iodouracil]. Culturing of ES cells and collection and injection of blastocysts have been previously described (Matzuk et al., 1992). For genomic Southern blot analysis, BglII-digested DNA was transferred to GeneScreen Plus nylon membrane and probed with an external 190 bp PCR synthesized fragment corresponding to exon 9 sequence (3′ probe). An internal 200 bp PCR synthesized fragment (49 bp exon 1 plus 150 bp 5′ upstream sequence) was also used to distinguish the wild-type and Npm2 null (Npm2^{tm1Zuk}) alleles when DNA was digested with BamH1. A PCR-synthesized probe containing the 137 bp exon 2 sequence was used to verify that exon 2 was absent in mice homozygous for the Npm2^{tm1Zuk} allele when DNA was digested with Pst1. A single correctly targeted ES cell clone (named Npm2-118-B11) was expanded, and ES cells were injected into C57B1/6 blastocysts to obtain chimeric mice which ultimately produced C57B16/129SvEv hybrid and 129 inbred F1 progeny.

Fertilized Egg Collection, Culture, and Fluorescent Staining

Immature (21-24 day old) females were injected intraperitoneally with 5 IU PMSG (pregnant mare serum gonadotropin) followed by injection of 5 IU hCG (human chorionic gonadotropin) to induce superovulation as described (Hogan et al., 1994; Matzuk et al., 1996). The injected mice were mated to wild-type male mice. Eggs were harvested the next morning from the oviducts of these mice. Cumulus cells were removed from the eggs by using 0.3 mg/ml hyaluronidase in M2 medium (Sigma). Eggs were cultured in M16 Medium (Sigma) covered with light paraffin oil in a humidified 37° C. incubator with an atmosphere of 5% CO₂ and 95% air (Hogan et al., 1994).

For the staining of DNA, eggs were washed once in PBS, incubated in 4% paraformaldehyde in PBS containing 10 ug/ml bisbenzimide (Hoechst 33258) for 20 min at room temperature, washed twice with PBS, mounted with Fluoromount-G, and photographed by using fluorescence microscopy (Axioplan 2 imaging, Carl Zeiss).

### EXAMPLE 9

### Npm2 Homology

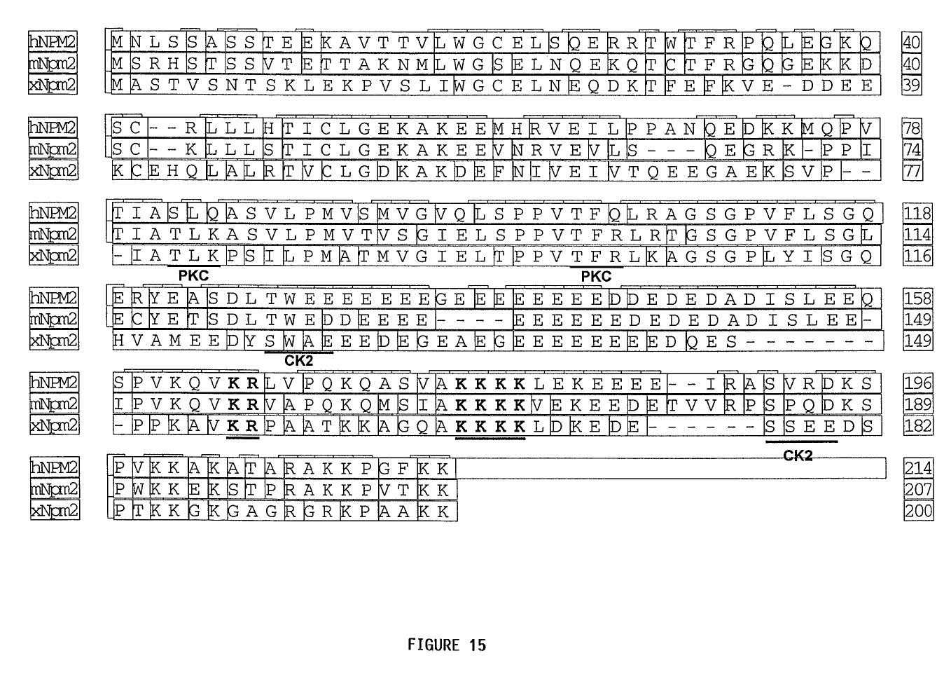

Upon primary screening of the mouse ovarian cDNA libraries, the O1-236 cDNA fragment detected 22 positive phage clones out of 300,000 screened. Two of these clones (236-1 and 236-3), which approximated the mRNA size and which were derived from the two independent libraries, were analyzed further by restriction endonuclease digestion and DNA sequence analysis. These independent clones form a 984 bp overlapping contig (excluding the polyA sequences) and encode a 207 amino acid open reading frame. Including the polyA tail, this sequence approximates the 1.0 kb mRNA seen by Northern blot analysis suggesting that nearly all of the 5′ UTR sequence has been isolated. When the nucleotide sequence is subjected to public database search, no significant matches were initially derived. However, database search with the 207 amino acid open reading frame demonstrated high homology with several nucleoplasmin homologs from several species. Interestingly, O1-236 shows highest homology with Xenopus laevis nucleoplasmin. At the amino acid level, O1-236 is 48% identical and 71% similar to Xenopus laevis nucleoplasmin (FIG. 15). Based on this homology and the expression patterns of both gene products in oocytes, the inventors have termed the gene Npm2 (mouse nucleoplasmin 2) since it is the mammalian ortholog of Xenopus laevis nucleoplasmin (called Xnpm2 in (MacArthur and Shackleford, 1997a); see below for discussion of nomenclature).

Using the Npm2 cDNA sequence to search the EST database, two human cDNA clones containing sequences homologous to the mouse Npm2 were found. Sequence analysis of these two ESTs was performed. The two independent clones form a 923 bp overlapping contig which encodes a 214 amino acid open reading frame. At the amino acid level, human NPM2 is 48% and 67% identical to Xnpm2 and mouse Npm2, respectably (FIG. 15).

When the nucleoplasmin 2 sequences from human, mouse, and Xenopus are compared, several interesting features are realized. Xenopus laevis nucleoplasmin has a bipartite nuclear localization signal consisting of KR-(X)₁₀-KKKK (Dingwall et al., 1987). Deletion of either of these basic amino acid clusters in nucleoplasmin prevents translocation to the nucleus (Robbins et al., 1991). When the three nucleoplasmin 2 sequences were compared, this bipartite sequence was shown to be 100% conserved among the three proteins (FIG. 15). Thus, Npm2 would be predicted to translocate to the nucleus.

Also conserved among the three nucleoplasmins is a long stretch of negatively charged residues (FIG. 15). Amino acids 129-152 of human NPM2, amino acids 125-144 of mouse Npm2, and amino acids 128-146 of Xnpm2 are mostly glutamic acid (E) and aspartic acid (D) residues, with 22 out of the 24 residues for human NPM2, 19 out of the 20 residues for mouse Npm2, and 16 out of the 19 residues for Xnpm2 aspartic acid or glutamic acid. This region of Xnpm2 has been implicated to bind the positively charged protamines and histones. Thus, a similar function for this acidic region of mammalian Npm2 would also be predicted (see below).

The last obvious feature of the three Npm2 sequences is the high number of serine and threonine residues. The human NPM2 has 18 serines and 12 threonines (i.e., 13.1% of the residues), the mouse Npm2 sequence contains 19 serines and 17 threonines (i.e., 17.2% of the residues), and Xnpm2 has 12 serine and 11 threonine residues (i.e., 11.5% of the residues). Multiple putative phosphorylation sites are predicted from the three nucleoplasmin sequences. Several putative phosphorylation sequences that are conserved among the three proteins are shown in FIG. 15. Phosphorylation of Xnpm2 is believed to increase its translocation to the nucleus and also its activity (Cotten et al., 1986; Leno et al., 1996; Sealy et al., 1986; Vancurova et al., 1995). Similarly, phosphorylation may also alter the activity of the mammalian nucleoplasmin 2 proteins. Thus, since nucleoplasms are ovary-specific at the mRNA level and share highest identity, the inventors conclude that Npm2 and NPM2 are the orthologs of Xnpm2.

### EXAMPLE 10

### Ovarian-specific Expression of Mouse Npm2

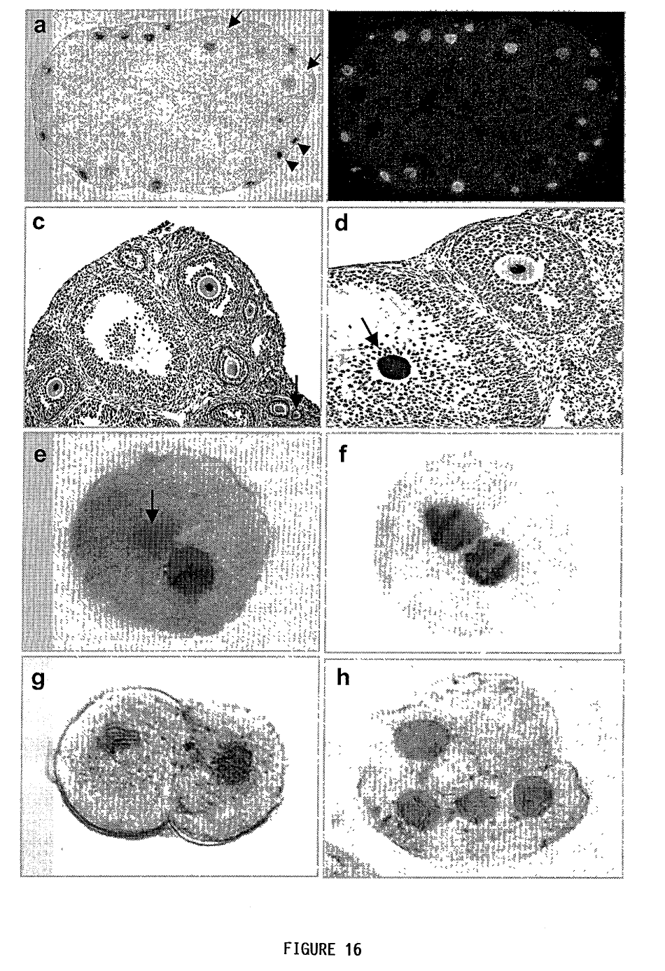

To define the cell-specific expression of the Npm2 gene product, in situ hybridization analysis was performed using wild-type mouse ovaries. The Npm2 gene product is oocyte-specific (FIGS. 16A and 16B). Npm2 is not expressed in oocytes of primordial (type 2) or small type 3a follicles (Pedersen and Peters, 1968)(data not shown) but is first detected in oocytes of intermediate-size type 3a follicles and all type 3b follicles (i.e., follicles with >20 granulosa cells surrounding the oocyte in largest cross-section). Expression of Npm2 mRNA persisted through the antral follicle stage. RT-PCR also showed that high levels of Npm2 mRNA persisted in ovulated oocytes and one cell embryos, but there was a dramatic reduction of the Npm2 mRNA in eight cell embryos and blastocysts (data not shown). Interestingly, the oocyte-specific expression pattern of Npm2 parallels the expression of other oocyte-specific genes which the inventors have studied including Gdf9 (Elvin et al., 2000; McGrath et al., 1995) and bone morphogenetic protein 15 (Dube et al., 1998).

The subcellular localization of Npm2 protein was determined by immunohistostaining of mouse ovaries with anti-Npm2 antibodies. A full-length recombinant Npm2 protein was produced in E. coli and injected into two goats to produce high titer antibodies. Consistent with the expression pattern of Npm2 mRNA, Npm2 protein was expressed in oocytes from type 3 to antral follicle stages. In randomly cycling mice, the anti-Npm2 antibody strongly and specifically stained the nucleus (FIG. 16C). The oocyte nucleus is also called the germinal vesicle (GV). The preovulatory surge of luteinizing hormone (LH) accelerates the maturation of GV oocytes and promotes GV breakdown (GVB). When mice were injected with PMSG and hCG to induce superovulation, the Npm2 protein redistributes in the oocytes of antral follicles after germinal vesicle breakdown. In preovulatory GVB oocytes, the Npm2 is evenly distributed in the cytoplasm of the oocyte (FIG. 16D). Since Xnpm2 has been implied to play a role in sperm DNA decondensation and pronuclei formation after fertilization, this redistribution suggests that the cytoplasmic Npm2 is now properly positioned to interact with the sperm nucleus at the time of fertilization. To examine the Npm2 expression after fertilization, early embryos were fixed, sectioned and stained with anti-Npm2 antibodies. In one-cell eggs, Npm2 begins to translocate back to the nucleus. FIG. 16E shows an intermediate stage in which one pronucleus is formed but other is not yet complete and some Npm2 is still present in the cytoplasm. At a later point (FIG. 16F), all of the Npm2 is present in the pronuclei. In two-cell (FIG. 16G) and eight-cell (FIG. 16H) embryos, the antibody continues to detect the Npm2 protein exclusively in the nucleus. Npm2 can continue to be detected at significantly reduced levels in blastocysts (embryonic day 3.5), but in embryonic day 6.5 embryos, Npm2 expression was undetectable.

### EXAMPLE 11

### Targeted Disruption of the Mouse Npm2 Gene and Generation of Npm2 Knockout Mice

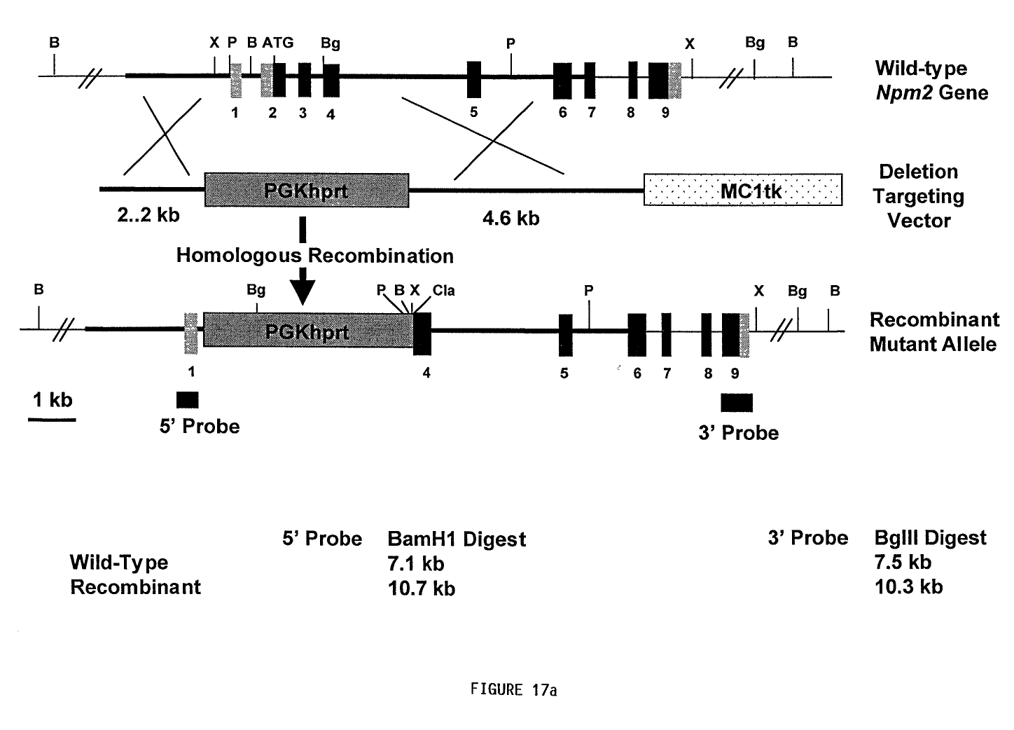

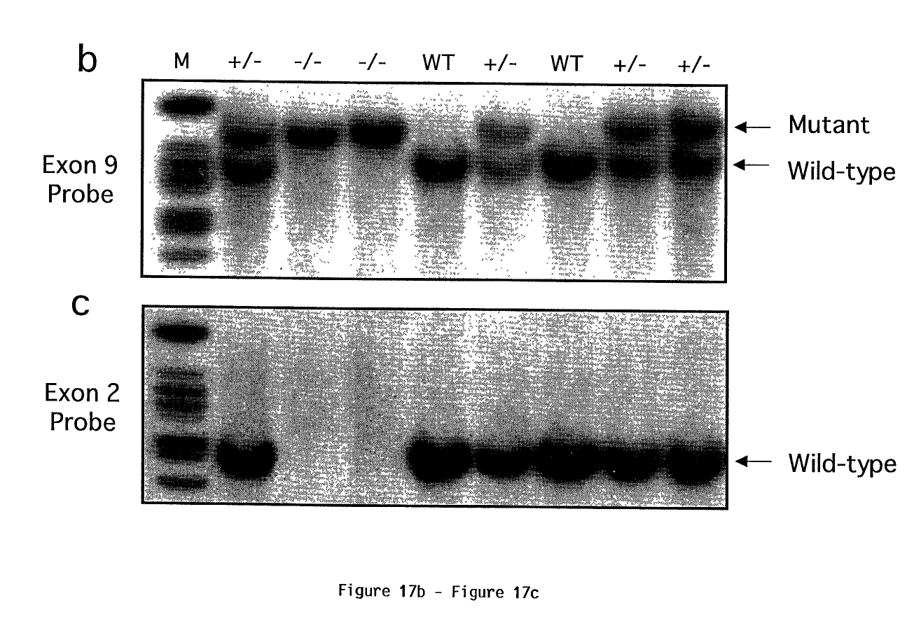

To study the role of Npm2 in mammalian oocyte development and early embryo development, the inventors disrupted the mouse Npm2 locus using ES cell technology. The targeting vector was constructed to delete exon 2 which contains the translation initiation codon and also exon 3 and the exon 4 splice junction (FIG. 17A). Outside of exon 2, only one other ATG is present in the remaining sequence (exon 6), and this ATG is positioned downstream of the acidic domain and between the bipartite nuclear localization consensus sequence. Thus, this vector generates an Npm2 null allele. F1 heterozygous (Npm2^{tm1Zuk/+}; herein called Npm2^{+/-}) mice were viable and fertile, and were intercrossed to investigate the developmental consequences of Npm2 deficiency. Genotype analysis of 230 F2 offspring from these intercrosses (FIG. 17B; Table 4) was consistent with a normal Mendelian ratio of 1:2:1, and a similar number of male and female homozygotes (Npm2^{-/-}) were produced. Therefore, Npm2 homozygous mutant male and female mice are viable and appear to have normal sexual differentiation demonstrating that Npm2 is not required during mouse embryogenesis.

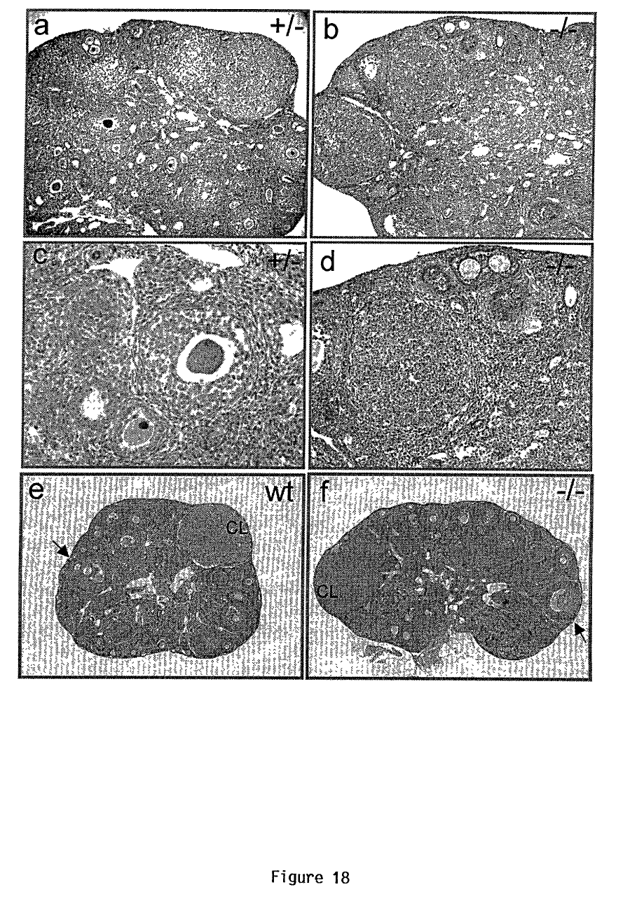

To confirm that the mice genotyped as Npm2 homozygotes lacked Npm2, a cDNA probe that could hybridize to exon 2 of the wild-type Npm2 gene was used for Southern blot analysis. As shown (FIG. 17C), this probe failed to detect any signal in DNA derived from homozygous (Npm2^{-/-}) mice in which exon 2 had been deleted. Furthermore, Npm2 immunohistochemical analysis was performed on Npm2 homozygotes and controls. Whereas the expression of Npm2 protein is noted in the ovaries from the heterozygous controls (FIGS. 18A and 18C), no protein is detected in oocytes in the homozygote ovaries (FIGS. 18B and 18D). This confirmed that the Npm2^{tm1Zuk} mutation is a null allele and that Npm2 homozygotes were completely deficient in Npm2 protein.

### EXAMPLE 12

### Loss of Npm2 Results in Female Infertility and Subfertility

To study the function of Npm2 in reproductive function, adult homozygous hybrid (C57B1/6/129SvEv) male or female mice were intercrossed with control hybrid mice (C57B1/6/129SvEv) mice. Consistent with the female-specific expression of Npm2 mRNA and protein, Npm2^{-/-} male mice are fertile and had no gross or histological defects in the testes (data not shown). Similarly, intercrosses of 10 female Npm2 heterozygotes with heterozygous males during a 5-8 month period resulted in 53 litters with 8.55 offspring/litter (0.97 litters/month)(Table 5). In contrast, only 9 out of 12 Npm2^{-/-} female mice became pregnant over a 5-8 month period resulting in 32 litters with an average of 2.75 offspring/litter (0.43 litters/month)(Table 5). Thus, deficiency of Npm2 leads to subfertility and infertility in females but not males.

### EXAMPLE 13

### Early Cleavage Defect in Npm-2-deficient Fertilized Eggs

To determine the causes of the fertility defects in the Npm2^{-/-} female mice, ovaries were first examined morphologically and histologically. There is no significant difference between Npm2^{-/-} and control ovaries at the gross or histological levels (FIGS. 18E and 18F). Normal folliculogenesis including the formation of corpora lutea were observed in the Npm2^{-/-} ovaries suggesting that ovulation occurred in these mice.

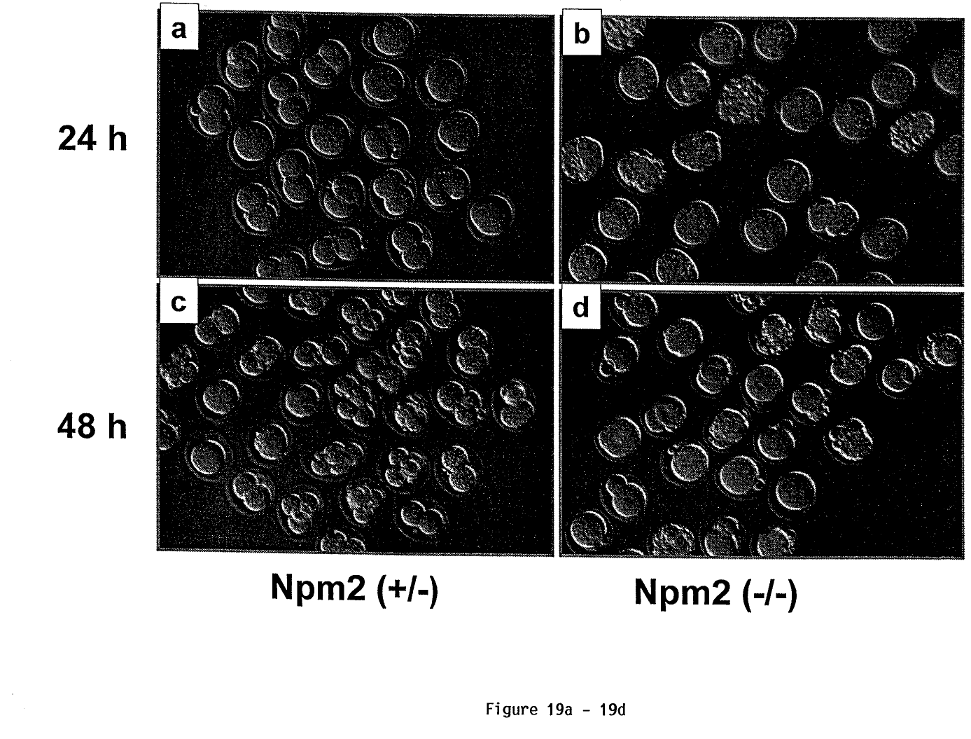

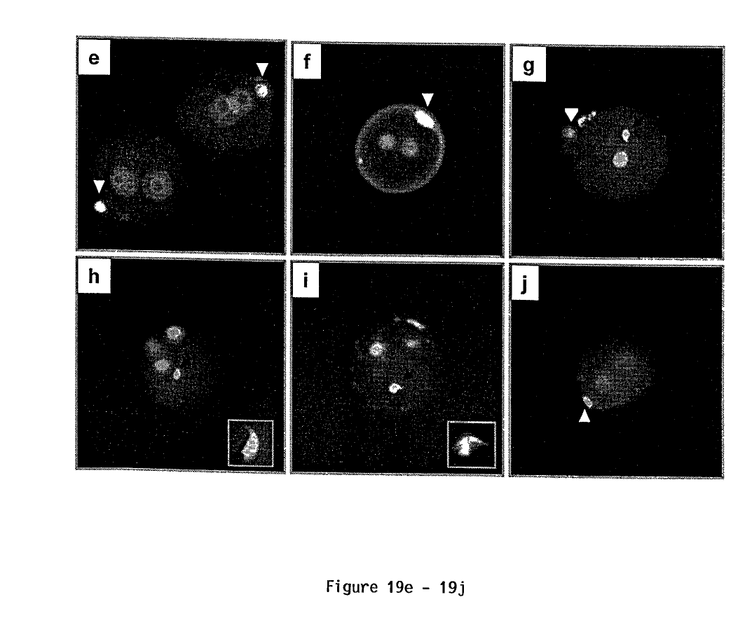

To confirm that ovulation was occurring and to further study the cause of the infertility and subfertility of the Npm2^{-/-} mice, pharmacological superovulation of wild-type, heterozygous, and homozygous mice was performed and the eggs were collected from the oviducts and cultured in vitro. Pregnant mare serum gonadotropin/human chorionic gonadotropin superovulation treatment of 21-24 day old mice resulted in similar numbers of eggs ovulated in Npm2^{-/-} females compared to wild-type or heterozygote controls (Table 6). The eggs from Npm2^{-/-} mice appear to be fertilized by spermatozoa normally because there were no significant differences between the Npm2^{-/-} and controls in the formation of the second polar body, evidence of fertilization. However, there was a substantial defect in the cleavage of one cell embryos into two cell embryos in the Npm2^{-/-} mice (Table 6, FIGS. 19A-19D). Besides a significant reduction in the number of two cell embryos, some bizarre, developmentally-abnormal embryos appeared during the 24 hours of in vitro culture (FIG. 19B). Unlike control eggs (FIG. 19C), Npm2^{-/-} eggs could not progress to the four cell embryo stage the current in vitro culture assay (FIG. 19D). Thus, the defect in the Npm2^{-/-} mice appeared to initiate after fertilization.

### EXAMPLE 14

### Defects in Sperm DNA Decondensation and Pronuclei Formation in Npm-2-deficient Eggs

Xenopus nucleoplasmin has been implicated to function in sperm DNA decondensation and pronuclei formation after fertilization. To define the potential mechanisms present in the mouse eggs that cause the defect in early cleavage of Npm2-deficient embryos, fertilized eggs isolated from superovulated mice that were crossed with wild-type male mice were stained with Hoechst 33258. As shown, most wild-type eggs (˜67.9%, 36/53) formed almost same size and equal density of the two pronuclei with one or two visible polar bodies around the cell edge during the time of collection (FIGS. 19E and 19F). Sperm nuclear DNA that had not decondensed in the wild-type eggs could not be found. In contrast, morphologically normal pronuclei were found in only ˜15.2% (14/92) of the Npm2^{-/-} eggs (FIG. 19J). The majority of the fertilized eggs from the Npm2^{-/-} mice displayed a highly condensed sperm head in the cyptoplasm (FIGS. 19G-19I). Besides the defect in the sperm DNA decondensation, the Npm2^{-/-} female pronucleus also seemed to be present in an overly condensed state in Npm2^{-/-} fertilized eggs (FIGS. 19G-19I). Thus, absence of Npm2 blocks sperm DNA decondensation in the majority of fertilized eggs and also results in defects in the decondensation of the female pronucleus.

### EXAMPLE 15

### Materials and Methods for Oo1

RNA Extraction and Northern Blot Analysis

Total RNA from mouse tissues was obtained by the RNA STAT-60 method (Leedo Medical Laboratories, Inc.). Agarose gel electrophoresis of RNA, its transfer to nylon membranes, and subsequent hybridization were performed by standard methods (Sambrook, et al., 1989).

In situ Hybridization

In situ hybridization was performed with the Oo1 specific probe. [α-³⁵S]UTP-labeled antisense and sense probes were generated by the Riboprobe T7/T3 combination system (Promega, Madison, Wis.). Hybridization was carried out as described previously (Albrecht, et al., 1997, Elvin et al., 1999A)

Mouse Ovary cDNA Library and Genomic Library Screening

1.1×10⁶ recombinant λ plaques were plated onto NZCYM bacterial lawn. Plaques were lifted onto duplicate Hybond-N nylon membrane filters (Amersham). The Oo1 cDNA probe fragment was used to screen wild-type mouse ovary cDNA libraries and the full-length Oo1 cDNA was used to screen a 129 SvEv mouse genomic library. Both probes were radiolabeled with [α-³²P]dCTP. Filters were prehybridized and hybridized as described previously (Matzuk and Bradley, 1992).

Chromosomal Mapping

The whole genome-radiation hybrid panel T31 (McCarthy et al., 1997) were purchased from Research Genetics (Huntsville, Ala.) and used according to the manufacturer's instruction. The panel was constructed by fusing irradiated mouse embryo primary cells (129aa) with hamster cells. Because the sequence of the hamster homologues for Oo1 is unknown, the inventors designed the reverse primers from the 3′-untranslated region of the murine sequence to minimize the risk of coamplification of the hamster homologues (Makalowski and Boguski, 1998). Oo1 gene specific primers were 5′-CTAGAAAAGGGGACTGTAGTCACT-3′ forward, and 5′-TGCATCTCCCACACAAGTCTTGCC-3′ reverse; pseudo Oo1 gene specific primers were 5′-CTAGAAAAGGGGACTATAGGCACC-3′ forward, and 5′-TGCATCTCTCACACAAGTGTTGCT-3′ reverse. Specificity of the two sets of primers was tested with A23 hamster DNA and 129 mouse DNA. The PCR reactions were performed in 15 μl final volume, containing 1 μl of each panel DNA, 1.25 u of Taq platinum DNA polymerase (Gibco, Rockville, Md.), companion reagents (0.25 mM dNTPs, 1.5 mM MgCl₂, 1× PCR buffer), and 0.4 μM of each primer. An initial denaturation step of 4 min at 94° C. was followed by amplification for 30 cycles (40 s at 94° C., 30 s at 60° C., and 30 s at 72° C.) and final elongation at 72° C. for 7 min.

### EXAMPLE 16

### Localization of Oo1 in Mouse Ovaries

Oocyte gene 1 (Oo1) (O1-180), a novel ovary-specific gene, encodes a 1.5 kb transcript. High-level expression of the Oo1 gene is detected in oocytes from the primary follicle through the antral follicle stage but not in oocytes of primordial follicles. The protein predicted from the cDNA ORF consists of 361 amino acids with a molecular mass of 40 kDa. It shows no homology to protein sequences in the public database. Oo1 contains a bipartite nuclear localization signal at positions 333 to 350, suggesting that it resides in the nucleus. Both the Oo1 gene and a related pseudogene contain four exons and three introns; the entire Oo1 gene is about 4.0 kb. Unlike Oo1, RT-PCR fails to detect the pseudogene transcripts in multiple adult mouse tissues. Using a mouse-hamster radiation hybrid panel, both Oo1 gene and pseudogene were mapped on mouse chromosome 5, which is syntenic with human 4p12.

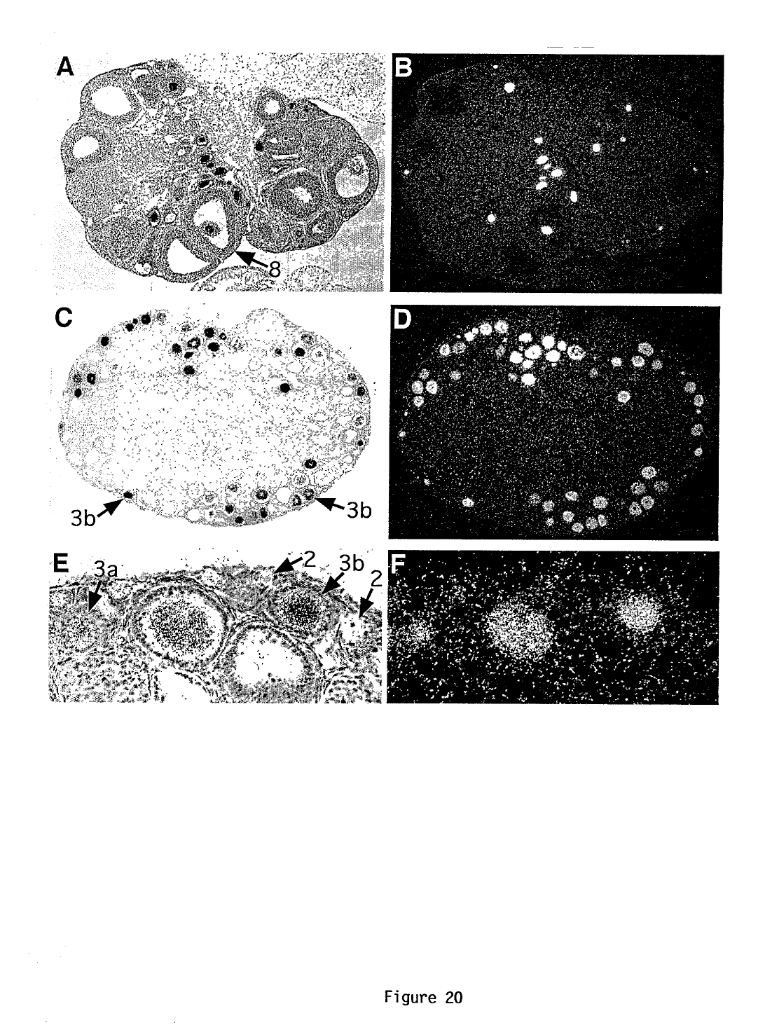

In situ hybridization showed high level expression of O1-180 (Oo1) localized to the oocytes within these ovaries. The expression of O1-180 within oocytes was evident at the one-layer (primary) follicle stage through the antral follicle stage, but no expression was observed at the primordial follicle stage. Because the number of follicles is increased in GDF-9-deficient ovaries due to the arrest of follicle development at the primary follicle stage, more O1-180 positive oocytes were detected in each section (FIG. 20).

### EXAMPLE 17

### Structure of the Oo1 Gene and Oo1 Pseudogene

A ZAP-express mouse ovary cDNA library was screened to isolate the full-length Oo1 cDNA. Excluding the polyA tail, the full-length Oo1 cDNA is about 1.3 kb, and encodes an open reading frame from nucleotides 26 to 1108. The Oo1 cDNA is homologous to several ESTs in the database, including ESTs in a mouse sixteen-cell embryo cDNA library (AU044294) and a mouse unfertilized egg cDNA library (AU023153). The polypeptide predicted from the Oo1 cDNA ORF consists of 361 amino acids, with a molecular mass of 40 kDa. Searching the public protein database failed to identify any known protein homologues. A bipartite nuclear localization signal was found at positions 333 to 350 (Lys-Arg-Pro-His-Arg-Gln-Asp-Leu-Cys-Gly-Arg-Cys-Lys-Asp-Lys-Arg-Leu-Ser), strongly suggesting that Oo1 is located in the oocyte nucleus.

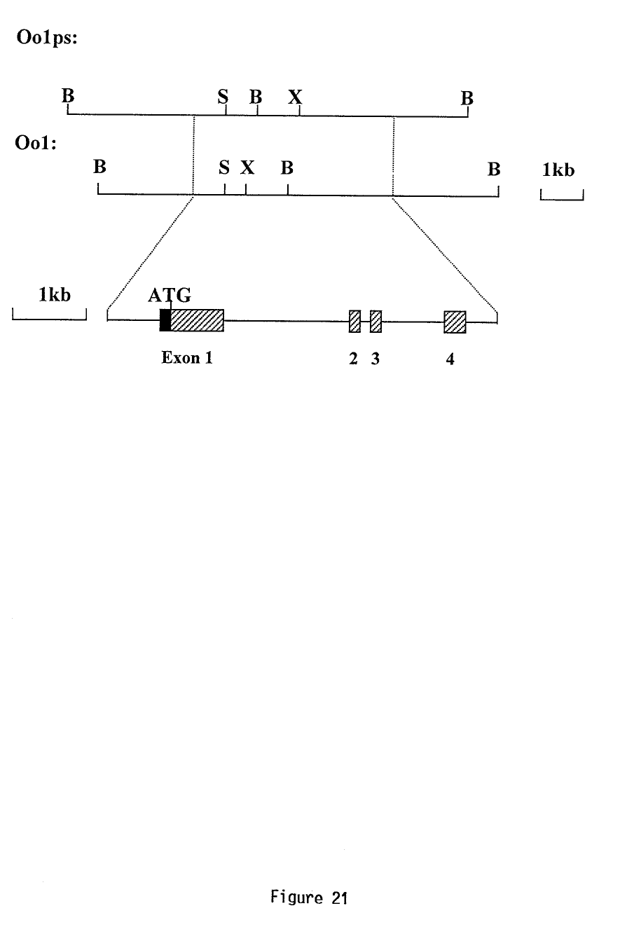

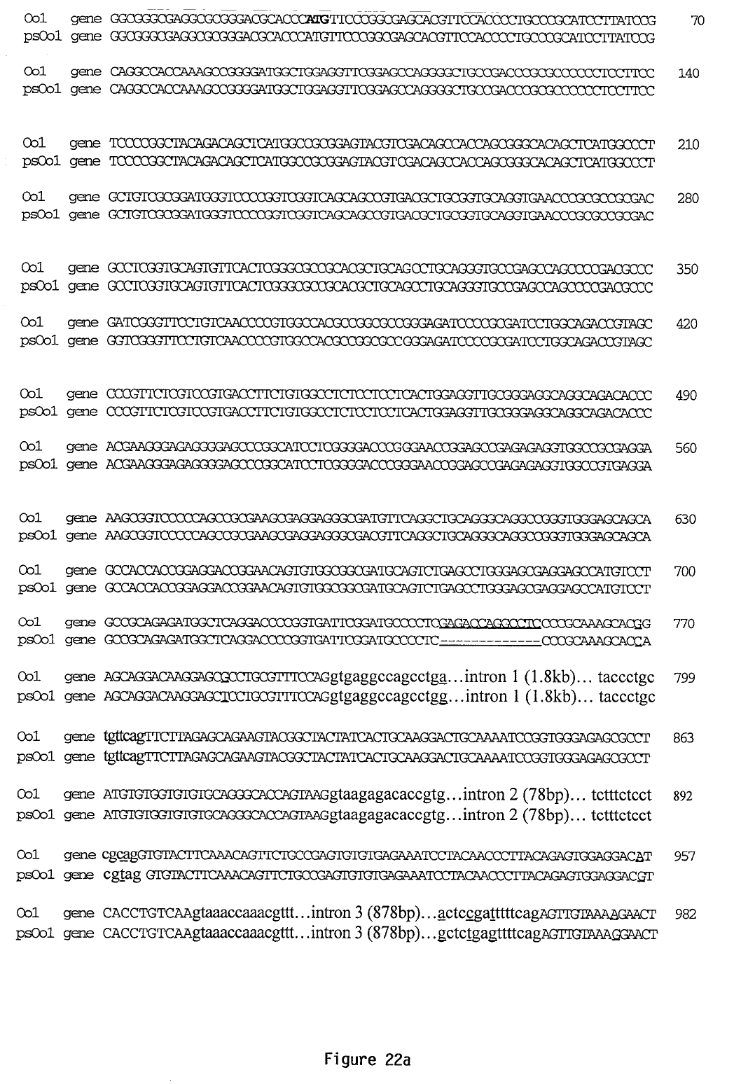

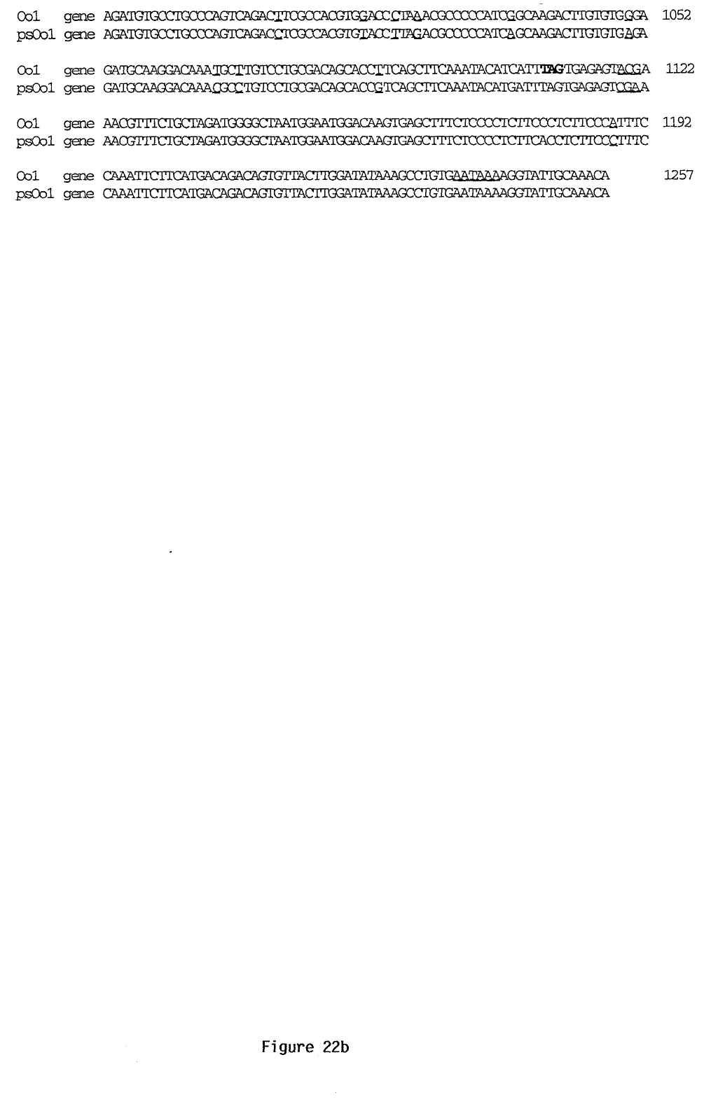

To clone the mouse Oo1 gene, a mouse genomic X Fix II phage library generated from mouse 129SvEv strain was screened with the full length Oo1 cDNA. Twelve independent λ recombinant clones were isolated; eight of which were identified as unique clones and were further characterized by subcloning, Southern blot analysis, and sequencing Surprisingly, only one genomic insert DNA starting 650 nucleotide upstream of exon 2 of the gene corresponded to the 3′-portion of the Oo1 gene. The remaining clones corresponded to a closely related gene, in which the exons share 98% identity with Oo1 cDNA. Based on the exon differences, Oo1 gene- and the related gene-specific primers were designed and reverse transcription-polymerase chain reactions (RT-PCR) were performed. cDNAs from 8-week-old C57 mouse tissues, including brain, heart, lung, spleen, liver, small intestine, stomach, kidney, uterus, testis, and ovary, were used as templates. Consistent with the Northern blot analysis, Oo1 cDNA was amplified exclusively in the ovary; while the related gene cDNA was not detectable in any of the tissues (data not shown). This indicates that the related gene isolated from the mouse genomic λ Fix II phage library is a pseudogene. A BAC 129SvJ mouse genomic library was screened by PCR with two sets of Oo1 gene-specific primers, and only one BAC clone was isolated. Sequencing of the entire coding region and exon-intron boundaries of the BAC and X phage clones showed that both the Oo1 and the Oo1 pseudogene contain four exons and three introns (FIG. 21). As shown in FIG. 22, all of the exon-intron boundaries satisfy the GT-AG intron donor-acceptor splice rule. The major difference between the Oo1 gene and the pseudogene is a 13-nt gap in exon 1 of the pseudogene, which the inventors expect results in a frame shift and early termination in exon 2 of the pseudogene. The sequences of exon 2 in both the Oo1 gene and pseudogene are identical, and there are single base pair mutations in exons 3 and 4 (FIG. 22) (SEQ ID NO: 18 to SEQ ID NO:25).

### EXAMPLE 18

### Mouse Chromosome 5

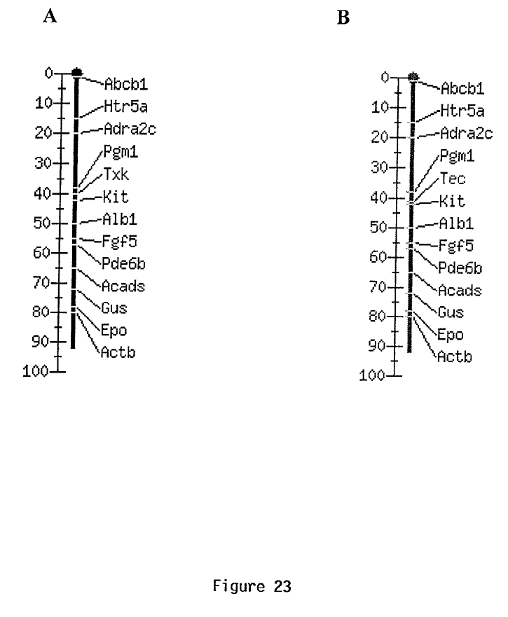

Both Oo1 gene and Oo1 pseudogene specific primers were designed respectively, and all 100 of the cell line DNAs of the T31 Mouse Radiation Hybrid Panel were screened by PCR in a duplicate assay. The data for each gene were submitted for analysis at the Jackson Laboratory Mouse Radiation Hybrid Mapper Server. Both genes were placed in the same region on mouse Chromosome 5. The Oo1 locus is at 40 cM, between two markers D5Buc48 and Txk, while the Oo1 pseudogene lies at 41 cM, between Tec and D5Mit356, just distal to the coding locus (FIG. 23.). This is syntenic to a region in humans Chromosome 4p12.

### EXAMPLE 19

### Targeted Disruption of the Mouse Oo1 Gene and Generation of Oo1 Knockout Mice

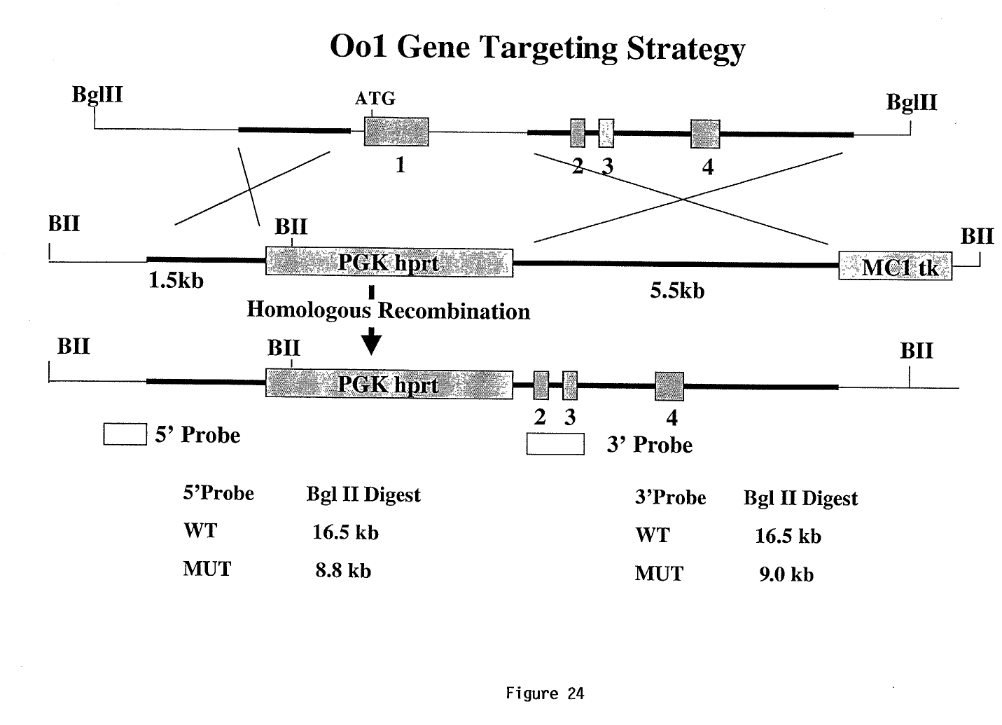

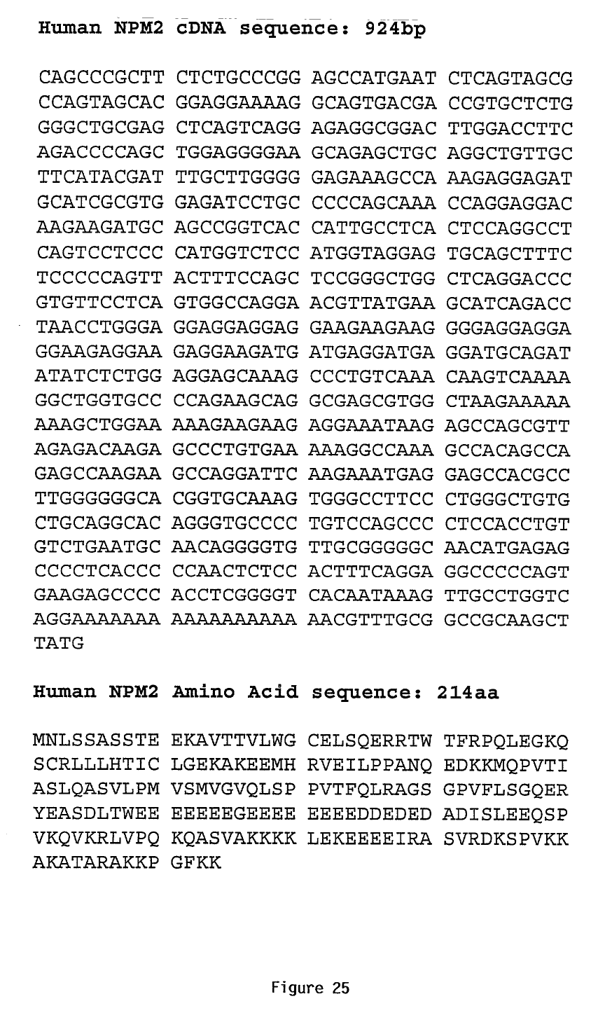

A targeting vector to mutate the Oo1 gene has been constructed from the isolated sequences described above (FIG. 24). To study the role of Oo1 in mammalian oocyte development and early embryo development, the inventors disrupted the mouse Oo1 locus using ES cell technology. The targeting vector was constructed to delete exon 1 which contains the translation initiation codon. Thus, this vector generates an Oo1 null allele.

TABLE 1
Summary of database searches of pO1 cDNA clones

| pO1 cDNA Matches | Number identified | Percentage |
|---|---|---|
| Known Genes | 180 | 54.4% |
| Mouse/Human EST | 120 | 36.2% |
| RARE ESTs (1 EST | (8) | (2.4%) |
| match) | | |
| ESTs from 2-cell library | (3) | (0.9%) |
| No match | 31 | 9.4% |
| Total | 331 | 100% |

TABLE 2
Analysis of ovarian cDNAs with no known function

| | | | | Further |
|---|---|---|---|---|
| | | | | studies |
| | | Upregulated | | (in situ |
| | | in GDF-9- | | hybridization; |
| PO1 | Adult mRNA | deficient | Database | chromosomal |
| cDNA | expression | ovary | match | mapping) |
| 24 | Multiple | No | — | No |
| 27 | Multiple | Yes | — | Oocyte- |
| | | | | specific by in |
| | | | | situ |
| 37 | Multiple | Yes | — | No |
| 70 | Multiple | No | — | No |
| 91 | | | 1 EST (2-cell) | |
| 97 | Multiple | No | ? | No |
| 101 | Multiple | Nol | — | No |
| 114 | Multiple | No | — | No |
| 110 | Multiple | Yes | — | No |
| 126 | Multiple | Yes | — | No |
| 180 | Ovary-specific | Yes | — | Oocyte- |
| | | | | specific by in |
| | | | | situ |
| 184 | Ovary-specific | Yes | >1 EST (All 2- | Oocyte- |
| | | | cell) | specific by in |
| | | | | situ |
| 186 | Ovary-specific | Yes | — | Granulosa |
| | | | | cell-specific by |
| | | | | in situ |
| 223 | Multiple | No | — | No |
| 224 | Multiple | No | — | No |
| 236 | Ovary-specific | Yes | 6 EST (2 c- | Oocyte- |
| | | | cell and others) | specific by in |
| | | | | situ |
| 255 | Multiple | No | “zinc-finger” | |
| | | | domains | |
| 279 | Multiple | No | — | No |
| 317 | Multiple | No | — | No |
| 330 | Multiple | No | — | No |
| 331 | Multiple | No | — | No |
| 332 | Multiple | No | — | No |
| 334 | Multiple | No | — | No |
| 371 | Multiple | No | — | No |

TABLE 3
Analysis of partial or full-length cDNAs

| pO1 cDNA | ORF | DataBase Homolog |
|---|---|---|
| 01-180 | 361 aa | No |
| 01-184 | 426 | No |
| 01-236 | 207 | Yes; Xenopus laevis |
| | | nucleoplosmin homolog |
| | | (81% similar) |

TABLE 4
Heterozygous mating

| | −/− | +/− | Wild type | Total |
|---|---|---|---|---|
| Male | 27 | 71 | 19 | 117 |
| Female | 27 | 53 | 33 | 113 |
| Total | 54 | 124 | 52 | 230 |
| % | 23 | 54 | 23 | 100 |

TABLE 5
Matings of Npm2 Knockout Mice
(129/C57 Mice; 5-8 Months of Breeding)

| | | | | | Average | |
|---|---|---|---|---|---|---|
| Genotype of Parents | | | | | Litter size | Litters/month |
| Male | | Female | Mothers | Litters | (Mean ± SEM) | (Mean ± SEM) |
| +/− | X | +/− | 8 | 51 | 8.55 ± 0.34* | 0.97 ± 0.03** |
| WT | X | −/− | 12*** | 32 | 2.75 ± 0.25* | 0.43 ± 0.10** |

*P < 0.001
**P < 0.005
***Three mice are infertile

TABLE 6
In vitro culture of eggs released by superovulation

| | Number | Eggs | Presence of | |
|---|---|---|---|---|
| Geno- | of | (Mean ± | polar body | 2 cell embryos |
| type | females | SEM) | (Mean ± SEM) | (Mean ± SEM (%) |
| Wild | 7 | 14.4 ± 3.8 | 8.6 ± 1.4 | 7.3 ± 2.1**(50.5%) |
| type | | | | |
| Npm2^{±} | 21 | 12.6 ± 2.1 | 6.9 ± 0.9 | 7.1 ± 1.3**(56.3%) |
| Npm2^{-/-} | 15 | 15.7 ± 3.9 | 7.2 ± 1.7 | 1.3 ± 0.4**(8.3%) |

*Percentage of 2 cell embryos developed from total eggs
**P < 0.001

References

The following references, to the extent that they provide exemplary procedural or other details supplementary to those set forth herein, are specifically incorporated herein by reference:

Albrecht, U., Eichele, G., Helms, J. A., and Lu, H. C. (1997). Visualization of gene expression patterns by in situ hybridization. In Molecular and Cellular Methods in Developmental Toxicology, G. P. Daston, ed. (Boca Raton, Fla., CRC Press), pp. 23-48.

Burglin, T., Mattaj, I., Newmeyer, D., Zeller, R., and Robertis, E. D. (1987). Cloning of nucleoplasmin from Xenopus laevis oocytes and analysis of its developmental expression, Genes Dev 1, 97-107.

Carabatsos M., Elvin J., Matzuk, M., Albertini, D., 1998. Characterization of oocyte and follicle development in growth differentiation factor-9-deficient mice. Dev. Biol. 203, 373-384.

Chan, W. Y., Liu, Q. R., Borjigin, J., Busch, H., Rennert, 0. M., Tease, L. A., and Chan, P. K. (1989). Characterization of the cDNA encoding human nucleophosmin and studies of its role in normal and abnormal growth, Biochemistry 28, 1033-9.

Channing, C. P., 1970. Influences of the in vivo and in vitro hormonal environment upon luteinization of granulosa cells in tissue culture. Recent Prog. Horm. Res. 26, 589-622.

Cotten, M., Sealy, L., and Chalkley, R. (1986). Massive phosphorylation distinguishes Xenopus laevis nucleoplasmin isolated from oocytes or unfertilized eggs, Biochemistry 25, 5063-5069.

Crevel, G., Huikeshoven, H., Cotterill, S., Simon, M., Wall, J., Philpott, A., Laskey, R. A., McConnell, M., Fisher, P. A., and Berrios, M. (1997). Molecular and cellular characterization of CRP1, a Drosophila chromatin decondensation protein, J Struct Biol 118, 9-22.

Dong, J., Albertini, D. F., Nishimori, K., Kumar, T. R., Lu, N., Matzuk, M. M., 1996. Growth differentiation factor-9 is required during early ovarian folliculogenesis. Nature 383, 531-535.

Dilworth, S., Black, S., and Laskey, R. (1987). Two complexes that contain histones are required for nucleosome assembly in vitro: role of nucleoplasmin and N1 in Xenopus egg extracts, Cell 51, 1009-1018.

Dimitrov, S., and Wolffe, A. (1996). Remodeling somatic nuclei in Xenopus laevis egg extracts: molecular mechanisms for the selective release of histones H1 and H1_from chromatin and the acquisition of transcriptional competence, EMBO Journal 15, 5897-5906.

Dingwall, C., Dilworth, S., Black, S., Kearsey, S., Cox, L., and Laskey, R. (1987). Nucleoplasmin cDNA sequence reveals polyglutamic acid tracts and a cluster of sequences homologous to putative nuclear localization signals, EMBO J 6, 69-74.

Dube, J. L., Wang, P., Elvin, J., Lyons, K. M., Celeste, A. J., and Matzuk, M. M. (1998). The bone morphogenetic protein 15 gene is X-linked and expressed in oocytes, Molecular Endocrinology 12, 1809-1817.

Earnshaw, W., Honda, B., and Laskey, R. (1980). Assembly of nucleosomes: the reaction involving X. laevis nucleoplasmin, Cell 21, 373-383.

El-Fouly, M. A., Cook, B., Nekola, M., Nalbandov, A. V., 1970. Role of the ovum in follicular luteinization. Endocrinology 87, 288-293.

Elvin, J. A., Clark, A. T., Wang, P., Wolfman N. M., Matzuk, M. M., 1999B. Paracrine actions of growth differentiation factor-9 in the mammalian ovary. Mol. Endocrinol. 13, 1035-1048.

Elvin, J. A., and Matzuk, M. M. (1998). Mouse models of ovarian failure, Reviews of Reproduction 3, 183-195.

Elvin, J. A., Yan, C., and Matzuk, M. M. (2000). Oocyte-expressed TGF-beta superfamily members in female fertility, Mol Cell Endocrinol 159, 1-5.

Elvin, J. A., Yan, C., Wang, P., Nishimori, K., and Matzuk, M. M. (1999). Molecular characterization of the follicle defects in the growth differentiation factor 9-deficient ovary, Mol Endocrinol 13, 1018-34.

Elvin, J. A., Yan, C., Matzuk, M. M., 2000. Growth differentiation factor-9 stimulates progesterone synthesis in granulosa cells via a prostaglandin E2/EP2 receptor pathway. Proc. Natl. Acad. Sci. USA, 97: 10288-10293.

Hogan, B., Beddington, R., Costantini, F., and Lacy, E. (1994). Manipulating the mouse embryo-a laboratory manual (Plainview, Cold Spring Harbor Laboratory Press).

Ito, T., Tyler, J. K., Bulger, M., Kobayashi, R., and Kadonaga, J. T. (1996). ATP-facilitated chromatin assembly with a nucleoplasmin-like protein from Drosophila melanogaster, J Biol Chem 271, 25041-8.

Iwata, K., Hozumi, K., Iihara, A., Nomizu, M., Sakairi, N., and Nishi, N. (1999). Mechanism of salmon sperm decondensation by nucleoplasmin, Int J Biol Macromol 26, 95-101.

Krohne, G., and Franke, W. (1980a). Immunological identification and localization of the predominant nuclear protein of the amphibian oocyte nucleus, Proc Natl Acad Sci 77, 1034-1038.

Kumar, T. (1994). The value and use of different contraceptive methods, Human Rep 9, 578-585.

Kumar, T. R., Wang, Y., Lu, N., and Matzuk, M. M. (1997). Follicle stimulating hormone is required for ovarian follicle maturation but not male fertility, Nature Genetics 15, 201-204.

Laskey, R., Mills, A., Philpoff, A., Leno, G., Dilworth, S., and Dingwall, C. (1993). The role of nucleoplasmin in chromatin assembly and disassembly, Philos Trans R Soc Lond B Biol Sci 339, 263-269.

Leno, G., Mills, A., Philpott, A., and Laskey, R. (1996). Hyperphosphorylation of nucleoplasmin facilitates Xenopus sperm decondensation at fertilization, J Biol Chem 271, 7253-7256.

MacArthur, C., and Shackleford, G. (1997a). Npm3: a novel, widely expressed gene encoding a protein related to the molecular chaperones nucleoplasmin and nucleophosmin, Genomics 42, 137-140.

MacArthur, C. A., and Shackleford, G. M. (1997b). Npm3: a novel, widely expressed gene encoding a protein related to the molecular chaperones nucleoplasmin and nucleophosmin, Genomics 42, 137-40.

Maeda, Y., Yanagimachi, H., Tateno, H., Usui, N., and Yanagimachi, R. (1998). Decondensation of the mouse sperm nucleus within the interphase nucleus, Zygote 6, 39-45.

Mahmoudi, M., and Lin, V. K. (1989). Comparison of two different hybridization systems in northern transfer analysis, Biotechniques 7, 331-332.

Matzuk, M. M., Finegold, M. J., Su, J.-G. J., Hsueh, A. J. W., and Bradley, A. (1992). α-Inhibin is a tumor-suppressor gene with gonadal specificity in mice, Nature 360, 313-319.

Matzuk, M. M., Kumar, T. R., and Bradley, A. (1995). Different phenotypes for mice deficient in either activins or activin receptor type II, Nature 374, 356-360.

Matzuk, M. M., Kumar, T. R., Shou, W., Coerver, K. A., Lau, A. L., Behringer, R. R., and Finegold, M. J. (1996). Transgenic models to study the roles of inhibins and activins in reproduction, oncogenesis, and development, Recent Prog Horm Res 51, 123-54.

McGrath, S. A., Esquela, A. F., and Lee, S.-J. (1995). Oocyte-specific expression of growth/differentiation factor-9, Molecular Endocrinology 9, 131-136.

McLay, D., and Clarke, H. (1997). The ability to organize sperm DNA into functional chromatin is acquired during meiotic maturation in murine oocytes, Dev Biol 186, 73-84.

Meric, F., Matsumoto, K., and Wolffe, A. (1997). Regulated unmasking of in vivo synthesized maternal mRNA at oocyte maturation. A role for the chaperone nucleoplasmin, J Biol Chem 272, 12840-12846.

Mills, A., Laskey, R., Black, P., and Robertis, E. D. (1980). An acidic protein which assembles nucleosomes in vitro is the most abundant protein in Xenopus oocyte nuclei, J Mol Biol 139, 561-568.

Nishimori, K., and Matzuk, M. M. (1996). Transgenic mice in the analysis of reproductive development and function, Reviews of Reproduction 1, 203-212.

Ohsumi, K., and Katagiri, C. (1991). Characterization of the ooplasmic factor inducing decondensation of and protamine removal from toad sperm nuclei: involvement of nucleoplasmin, Dev Biol 148, 295-305.

Pedersen, T., and Peters, H. (1968). Proposal for a classification of oocytes and follicles in the mouse ovary, Journal of Reproduction and Fertility 17, 555-557.

Perreault, S. (1992). Chromatin remodeling in mammalian zygotes, Mutat Res 296, 43-55.

Philpott, A., and Leno, G. (1992). Nucleoplasmin remodels sperm chromatin in Xenopus egg extracts, Cell 69, 759-767.

Philpott, A., Leno, G., and Laskey, R. (1991). Sperm decondensation in Xenopus egg cytoplasm is mediated by nucleoplasmin, Cell 65, 569-578.

Robbins, J., Dilworth, S., Laskey, R., and Dingwall, C. (1991). Two interdependent basic domains in nucleoplasmin nuclear targeting sequence: identification of a class of bipartite nuclear targeting sequence, Cell 64, 615-623.

Schmidt-Zachmann, M. S., and Franke, W. W. (1988). DNA cloning and amino acid sequence determination of a major constituent protein of mammalian nucleoli. Correspondence of the nucleoplasmin-related protein NO38 to mammalian protein B23, Chromosoma 96, 417-26.

Sealy, L., Cotten, M., and Chalkley, R. (1986). Xenopus nucleoplasmin: egg vs. oocyte, Biochemistry 25, 3064-3072.

Senapathy, P., Shapiro, M., and Harris, N. (1990). Splice junctions, branch point sites, and exons: sequence statistics, identification, and applications to genome project, Methods Enzymol 183, 252-278.

Service, R. (1996). Panel wants to break R&D barrier, Science 272, 1258.

Vanderhyden, B. C., Cohen, J. N., Morley, P., 1993. Mouse oocytes regulate granulosa cell steroidogenesis. Endocrinology 133, 423-426.

Vancurova, I., Paine, T., Lou, W., and Paine, P. (1995). Nucleoplasmin associates with and is phosphorylated by casein kinase II, J Cell Sci 108, 779-787.

We claim:

1. Substantially pure O1-236 (Npm2) having the amino acid sequence set forth in FIG. 2 (SEQ ID NO: 17).

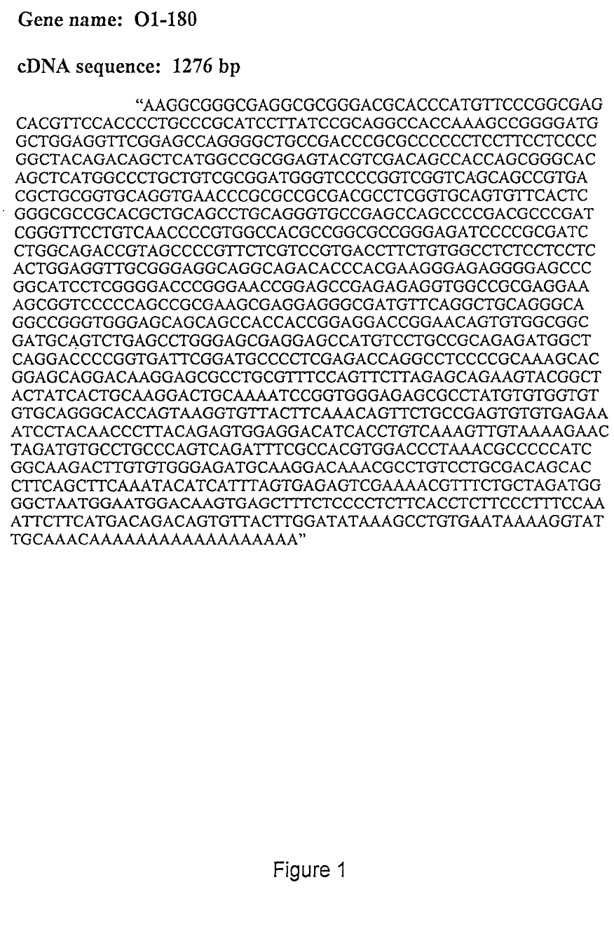

2. An isolated polynucleotide having the polynucleotide sequence set forth in FIG. 1 (SEQ ID NO: 16).

3. A transgenic mouse comprising a defect in formation of early cleavage embryos caused by a disruption of its genome in the O1-236 (Npm2) gene.

4. The transgenic mouse of claim 3 wherein said disruption is a homozygous disruption.

5. The transgenic mouse of claim 3 wherein the defect is due to a failure of sperm DNA decondensation in fertilized eggs.

6. The transgenic mouse of claim 3 wherein the defect is due to defective decondensation of the female pronucleus in fertilized eggs.

7. The transgenic mouse of claim 3 wherein said disruption consists of a deletion of exon 2, exon 3 and the exon 4 splice junction.

8. The method of making a transgenic mouse comprising a disruption of its genome in the O1-236 (Npm2) gene, comprising the steps of:
(a) introducing an O1-236 (Npm2) targeting vector comprising a PGK-hprt expression cassette into a mouse embryonic stem cell;
(b) selecting for the mutation of the O1-236 (Npm2) gene in embryonic stem cells;
(c) introducing said mouse embryonic stem cells with the mutation of the O1-236 (Npm2) gene into a mouse blastocyst;
(d) transplanting said mouse blastocyst into a pseudopregnant mouse;
(e) allowing said transplanted mouse blastocyst to develop to term; and
(f) identifying a transgenic mouse comprising a disruption of its genome in the O1-236 (Npm2) gene in at least one allele.

9. The method of claim 8 further comprising the step of breeding two transgenic mice to obtain a transgenic mouse comprising a homozygous disruption of its genome of the (O1-236) Npm2 gene.

10. The method of claim 9 wherein said disruption results in said transgenic mouse exhibiting a defect in formation of early cleavage embryos.

11. A transgenic mouse comprising a disruption of its genome in the O1-180 (Oo1) gene consisting of a deletion of exon 1.

12. The transgenic mouse of claim 11 wherein said disruption is a homozygous disruption.

13. The method of making a transgenic mouse comprising a disruption of its genome in the O1-180 (Oo1) gene, comprising the steps of:
(a) introducing an O1-180 (Oo1) targeting vector comprising a PGK-hprt expression cassette into a mouse embryonic stem cell;
(b) selecting for the mutation of the O1-180 (Oo1) gene in embryonic stem cells;
(c) introducing said mouse embryonic stem cells with the mutation of the O1-180 (Oo1) gene into a mouse blastocyst;
(d) transplanting said mouse blastocyst into a pseudopregnant mouse;
(e) allowing said transplanted mouse blastocyst to develop to term; and
(f) identifying a transgenic mouse comprising a disruption of its genome in the O1-180 (Oo1) gene in at least one allele.

14. The method of claim 13 further comprising the step of breeding two transgenic mice to obtain a transgenic mouse comprising a homozygous disruption of its genome of the O1-180 (Oo1) gene.

15. The method of making a transgenic mouse comprising a disruption of its genome in the O1-184 gene, comprising the steps of:
(a) introducing an O1-184 targeting vector comprising a PGK-hprt expression cassette into a mouse embryonic stem cell;
(b) selecting for the mutation of the O1-184 gene in embryonic stem cells;
(c) introducing said mouse embryonic stem cells with the mutation of the O1-184 gene into a mouse blastocyst;
(d) transplanting said mouse blastocyst into a pseudopregnant mouse;
(e) allowing said transplanted mouse blastocyst to develop to term; and
(f) identifying a transgenic mouse comprising a disruption of its genome in the O1-184 gene in at least one allele.

16. The method of claim 15 further comprising the step of breeding two transgenic mice to obtain a transgenic mouse comprising a homozygous disruption of its genome of the O1-184 gene.

17. The method of making a transgenic mouse comprising a disruption of its genome in more than one of the O1-236 (Npm2), O1-180 (Oo1) or O1-184 genes, comprising the steps of:
(a) introducing an O1-236 (Npm2), O1-180 (Oo1) or O1-184 targeting vector comprising a PGK-hprt expression cassette into a mouse embryonic stem cell;
(b) selecting for the mutation of the O1-236 (Npm2), O1-180 (Oo1) or O1-184 gene in embryonic stem cells;
(c) introducing said mouse embryonic stem cells with the mutation of the O1-236 (Npm2), O1-180 (Oo1) or O1-184 gene into a mouse blastocyst;
(d) transplanting said mouse blastocyst into a pseudopregnant mouse;
(e) allowing said transplanted mouse blastocyst to develop to term; and
(f) identifying a transgenic mouse comprising a disruption of its genome in the O1-236 (Npm2), O1-180 (Oo1) or O1-184 gene in at least one allele.

18. The method of claim 17 further comprising the step of breeding two transgenic mice to obtain a transgenic mouse comprising a homozygous disruption of its genome of the O1-236 (Npm2), O1-180 (Oo1) or O1-184 genes.
